(19) United States
(12) Patent Application Publication
(10) Pub. No.: US 2023/0398234 A1
(43) Pub. Date: Dec. 14, 2023

(54) NOVEL FUNGAL COMPOIND FORMULATIONS AND THEIR THERAPEUTIC METHODS OF USE

(71) Applicant: Mydecine Innovations Group Inc., Denver, CO (US)

(72) Inventor: Robert F. Roscow, Longmont, CO (US)

(21) Appl. No.: 18/032,922

(22) PCT Filed: Oct. 28, 2021
(86) PCT No.: PCT/US21/57017

§ 371 Date: Apr. 20, 2023

Related U.S. Application Data
(60) Provisional application No. 63/106,606, filed on Oct. 28, 2020.

(51) Int. Cl. A61K 47/69; A61K 47/60; A61K 36/068; A61K 36/074; A61K 36/07

(52) U.S. Cl. CPC A61K 47/6949; A61K 47/60; A61K 36/068; A61K 36/074; A61K 36/07

(57) ABSTRACT

The present invention further relates generally to novel systems, methods, and compositions for generating pharmaceutical compositions and formulations that include one or more fungal compounds and/or extracts.

### CROSS REFERENCE TO RELATED APPLICATIONS

This International PCT application claims the benefit of and priority to U.S. Provisional Application No. 63/106,606 filed Oct. 28, 2021, the specification, claims and drawings of which are incorporated herein by reference in their entirety.

### TECHNICAL FIELD

The present invention is directed to novel compositions and formulations including one or more fungal derived compounds and/or extracts. The present invention further relates generally to novel systems, methods, and compositions for generating water-soluble compositions of matter and formulations that include one or more fungal derived compounds and/or extracts. The present invention further relates generally to novel systems, methods, and compositions for increasing the bioavailability of one or more fungal derived compounds and/or extracts.

### BACKGROUND

Scientific research over the last several decades has identified numerous active compounds in fungi that have relevance to human nutrition and supplementation, as well as a variety of psychological and therapeutic applications. As a large group of diverse compounds, there are common issues inherent to their delivery during human and animal consumption. For example, many compounds of interest derived from fungal extracts are hydrophobic small molecules and, as a result exhibit poor solubility. In addition to problems with solubility, many of these fungal compounds of interests are highly-susceptible degradation in the gastro-intestinal (“GI”) tract and exhibit poor bioavailability. As such, there exists a long felt need for a safe and effective method to solubilize fungal derived compounds of interest, or otherwise protect the compounds from early degradation and metabolism to facilitate transport, storage, and adsorption through certain tissues and organs upon ingestion.

As detailed below, in one preferred embodiment, one or more fungal compounds of interest may be combined with one or more excipients to create nanoencapsulated complexes that increase overall solubility and/or bioavailability of the compounds, while also protecting them against early GI-tract degradation and first-pass metabolism processes. In a preferred embodiment, such nanoencapsulated complexes including a compound of interest, or one or more compounds or components of a fungal extract and Vitamin E TPGS that may be at, or below 20 nm in diameter, which is generally considered a threshold for effective bioavailability and mucosal membrane permeability of compounds, such as those of the invention. Such enhanced characteristics allow for the compounds of interest to be more effectively applied to human nutritional consumption, and more importantly in a variety of therapeutic application. As discussed below, the solubilized fungal compounds of interest may exhibit enhanced therapeutic effects, including enhanced potency, deceased effective doses, faster onset times, as well as systemic delivery throughout the body resulting from the novel compositions of the invention being able to pass through dermal, cellular, and mucus membranes, thus bypassing normal metabolism. This can be especially important for fungal compounds of interest that pass through the blood-brain barrier and act on select neuroreceptors in the brain producing a variety of therapeutic neurological effects.

### SUMMARY OF THE INVENTION

The present invention includes novel systems, methods, and compositions to generate solubilized nanoemulsion complexes containing one or more bioactive compounds of interest derived from a fungal extract. In one aspect, a solubilizing excipient, and preferably Vitamin E TPGS, may be used to create nanoemulsions and/or nanoencapsulated complexes (the terms being generally interchangeable as used herein) of the compounds of interest. In this preferred aspect, Vitamin E TPGS may be formulated with a compound or compounds derived, purified, or extracted from a select fungal strain or combination of fungal strains or fractions and form a nanoemulsion and/or nanoencapsulated complexes containing one or more fungal compounds of interest that may further exhibit one or more enhanced physiological or therapeutic characteristics, including but not limited to: 1) increase solubility; 2) increased bioavailability in a subject thereof; 3) increased resistance to degradation, and in particular increased resistance to degradation in the gastro-intentional (GI) tract of a subject thereof; 4) increased ability to cross cell and mucous membranes in a subject; and 5) increased ability to cross dermal layers in a subject thereof. Example compounds of interest include, but are not limited to: ganodermic acids, cordycepin, hericenones, erinacines, psilocybin, psilocin, baeocystin, norbaeocystin, psilacetin, and other natural tryptamines and compounds found in fungal feedstock.

In another aspect, an excipient, such as Vitamin E TPGS may be formulated with a compound or compounds found in a feedstock, and preferably a fungus species or genus to increase the solubility and bioavailability of poorly water soluble lipophilic compounds, and one or more pharmaceutically acceptable carriers, such as an aqueous solution. In one preferred aspect, an excipient, such as Vitamin E TPGS is formulated with a compound or compounds found in extracts of one or more fungal species selected from the group consisting of: Ganoderma sp., Cordyceps sp., Psilocybe sp, Hericium sp., Chaga sp., or a combination of the same.

In one aspect the invention is directed to the generation of a novel aqueous formulation containing a plurality of nanoencapsulated complexes containing a compound, or a fungal extract of interest. In this aspect, a first quantity of purified compound, or a fungal extract containing one or more compounds of interest obtained from a feedstock. This first quantity of the compound or extract of interest may be mixed with a first quantity of TPGS in an aqueous mixture which may be further agitated, such as through sonication, and undergo one or more rounds of heat cycling forming homogeneous aqueous solution.

In another aspect, the invention includes novel methods of generating a Vitamin E TPGS nanoencapsulated compound in the form of a solubilizable dry powder. In this aspect an aqueous formulation comprising a mixture of Vitamin E TPGS and a quantity of a compound or fungal extract of interest creating a plurality of micelles which is subsequently frozen and subjected to low pressure environment thereby removing water from the aqueous formulation creating a solubilizable dry powder containing the nanoencapsulated compounds.

In one aspect, the present fungal extracts or compounds may be combined with a suitable excipient, such as Vitamin E TPGS forming a solubilized nanoemulsion complex, which may be used to treat, prevent, inhibit, ameliorate and/or alleviate one or more disease conditions by administering a therapeutically effective amount of the solubilized nanoemulsion complex to a subject in need of such treatment, prevention, inhibition amelioration and/or alleviation. In another aspect of the invention, the solubilized nanoemulsion complexes may be configured to deliver consistent and rapid uptake of the compound(s) of interest in the body which may result in consumers ingesting a moderated and appropriate dose of the bioactive compound-infused product which may further provide a more predictable therapeutic, psychological, and/or psychotropic experience.

Additional aspects of the invention may become evident based on the specification and figures presented below.

### BRIEF DESCRIPTION OF THE FIGURES

FIG. 1: Concentrations of psilocybin in test & control solutions over a 4-day period.

FIG. 2: Concentrations of cordycepin in test & control solutions for variable mass ratios.

FIGS. 3A and 3B: (A) Chromatogram displaying three replicates of 15:1 mass ratio for the test and control Reishi solutions. The data indicates the existence of TPGS increases the solubility of extract components. (B) Chromatogram displaying three replicates of 15:1 mass ratio for the test and control Reishi solutions. The data indicates the existence of TPGS may increase the solubility of the extract components.

FIGS. 4A and 4B: (A) Chromatogram displaying an overlay of the replicates of 1 ml extract suspension: 1 ml TPGS solution for the three test and three control Lion's Mane solutions. The data indicates the existence of TPGS increases extract overall solubility. TPGS in solution is indicated by the large peak near minute 13. (B) Chromatogram displaying an overlay of the replicates of 1 ml extract suspension: 1 ml TPGS solution for the three test and three control Lion's Mane solutions. The data indicates the existence of TPGS increases extract overall solubility. TPGS in solution is indicated by the large peak near minute 13.

FIG. 5: The results of DLS for Cordyceps, Reishi & Lion's Mane Samples. Diameter of micelles appears to be independent of mass ratio.

### DETAILED DESCRIPTION OF THE INVENTION

Water-soluble Vitamin E-active polyethylene glycol esters of tocopheryl acid such as succinates were developed to provide water-soluble molecules having high vitamin E activity via either oral or parenteral administration. Examples include the polyethylene glycol acid succinate of α-tocopherol, known as d-α-tocopheryl polyethylene glycol succinate (TPGS). U.S. Pat. No. 2,680,749 discloses TPGS molecules in which the polyethylene glycols have average molecular weights of 400, 1000, and those varying between 600 and 6000. TPGS molecules in which the polyethylene glycol chains have an average molecular weight (MW) of about 1000 (TPGS 1000; available from Eastman Chemical Company, Kingsport, Tennessee) are currently used in oral pharmaceutical applications to enhance the bioavailability of various drugs. Due to the amphiphilic nature of TPGS 1000, incorporating TPGS 1000 into pharmaceutical formulations may enhance bioavailability by solubilizing some hydrophobic drugs.

As specifically used herein, the term “Vitamin E TPGS,” also interchangeably referred to as “TPGS,” refers to the esterification of Vitamin E succinate with polyethylene glycol 1000 resulting in the following structural formula:

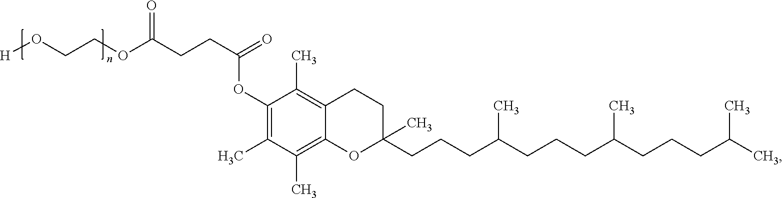

where “n” is an integer. As further used herein, the abbreviation TPGS can also refers to d-α tocopheryl polyethylene glycol 1000 succinate.

In one preferred embodiment, an excipient, such as Vitamin E TPGS is formulated with a compound or compounds found in a feedstock, and preferably a fungus species or genus to increase the solubility and bioavailability of poorly water soluble lipophilic compounds. In one preferred embodiment, an excipient, such as Vitamin E TPGS is formulated with a compound or compounds found in extracts of one or more fungal species selected from the group consisting of: Ganoderma sp., Cordyceps sp., Psilocybe sp, Hericium sp., Chaga sp., or a combination of the same.

As noted below, in a preferred embodiment, TPGS is formulated with a compound or compounds derived, purified, or extracted from a select fungal strain. In the context of the disclosure, TPGS may form a nanoemulsion and/or nanoencapsulated complexes containing one or more fungal compounds of interest that may further exhibit one or more enhanced physiological or therapeutic characteristics, including but not limited to: 1) increase solubility; 2) increased bioavailability in a subject thereof 3) increased resistance to degradation, and in particular increased resistance to degradation in the gastro-intentional tract of a subject thereof; 4) increased ability to cross cell and mucous membranes in a subject; and 5) increased ability to cross dermal layers in a subject thereof.

In one preferred embodiment, a first quantity of purified compound, or a fungal extract containing one or more compounds of interest, is first obtained from a feedstock. As used herein, “feedstock” generally refers to raw fungal material, comprising whole fungus alone, or in combination with one or more constituent parts or stages of a fungus comprising fruit bodies, mycelia, and spores, wherein the fungus or constituent parts may comprise material that is raw, dried, steamed, heated or otherwise subjected to physical processing to facilitate processing, which may further comprise material that is intact, cut, chopped, diced, milled, ground or otherwise processed to affected the size and physical integrity of the plant material. Occasionally, the term “feedstock” may be used to characterize an extraction product that is to be used as feed source for additional extraction processes. In other embodiment, “feedstock” may be used to characterize the biosynthesis of one of more compounds of interest found in a fungal extract from an initial set of starting chemical materials.

This first quantity of said compound or extract of interest may be mixed with a first quantity of TPGS in an aqueous mixture. In one embodiment, solubilization the mixture is initiated, for example through heat cycling, wherein the temperature is raised until the solid TPGS begins to dissolve. Such temperature being above room temperature. The mixture is then agitated until TPGS is dissolved and the viscosity of the mixture increases to a predetermined level. Next, the temperature applied to the mixture is lowered, for example to room temperature generating a homogeneous aqueous solution with lowered viscosity. If a homogeneous mixture is not achieved, then the heat cycling step may be repeated and/or the temperature and agitation time are increased.

As used herein, the step of obtaining “a first quantity of purified compound, or a fungal extract containing one or more compounds of interest . . . from a feedstock” refers to isolating a compound of extract, for example an extract from one or more separate fractions, from the rest of the fungal material, separation can be done by a number of techniques known in the art. For example, thin layer chromatography, high performance liquid chromatography, gas chromatography, electrophoresis, microscopy, supercritical fluid chromatography, etc. As noted above, in certain embodiment, a feedstock may be processed so as to produce different fractions from which a compound or extract may be taken. As used herein, the term “fraction” means the extraction comprising a specific group of chemical compounds characterized by certain physical, chemical properties or physical or chemical properties. Examples may include, but not be limited to: 1) an essential oil fraction comprises lipid soluble, water insoluble compounds; 2) triterpene fraction comprises the water-soluble and ethanol soluble triterpene compounds; and 4) a polysaccharide fraction comprises water soluble-ethanol insoluble polysaccharide compounds; 5) a protein fraction containing peptide constituents.

The ratio of compound or extract to Vitamin E TPGS may be variable based on the chemical properties of the compound or extract, the intended use of the nanoemulsion, as well as any therapeutic considerations such as dosage. In one embodiment, the compositions disclosed herein comprise a ratio of Vitamin E TPGS to the first quantity of a compound or fungal extract of interest of about: 1:1 to 1:10,000; 1:1 to 1:1000; 1:1 to 1:100; 1:1 to 1:10 by percent mass; 1:1 to 1:8 by percent mass; 1:1 to 1:6 by percent mass; 1:1 to 1:4 by percent mass; greater than 1:10 by percent mass.

In one preferred embodiment, the invention includes a novel method of making an aqueous formulation comprising adding between 0.1-99% water, by mass percent, to a composition comprising a first compound or fungal extract of interest and Vitamin E TPGS. As used herein, the term “aqueous formulation” refers to a solution wherein a first compound or fungal extract of interest and Vitamin E TPGS are dispersed throughout water and wherein the water acts as a solvent. In one embodiment, the aqueous formulation is made by methods disclosed herein. In one embodiment, the aqueous formulation comprises a second compound or fungal extract of interest.

In one embodiment, the aqueous formulation comprises a third compound or fungal extract of interest. In one embodiment, the aqueous formulation comprises a plurality of compounds or fungal extracts of interest.

In one preferred embodiment, water accounts for between 0.1-10% of the mass percent of the aqueous formulation. In another embodiment, water accounts for between 0-5% of the mass percent of the aqueous formulation. In another embodiment, water accounts for between 0-3% of the mass percent of the aqueous formulation. In another embodiment, water accounts for less than 1% of the mass percent of the aqueous formulation. In another embodiment, water accounts for greater than % of the mass percent of the aqueous formulation.

In another preferred embodiment, the invention includes novel methods of generating a Vitamin E TPGS nanoencapsulated compound in the form of a solubilizable dry powder. In one embodiment, the method may include one or more of the following steps:
incubating compound or fungal extract of interest derived from a fungal feedstock in a pharmaceutically acceptable carrier having a quantity of TPGS; and
sonicating said pharmaceutically acceptable carrier, wherein said compound or fungal extract of interest and said TPGS form a nanoencapsulated complex, and where said pharmaceutically acceptable carrier is an aqueous solution;
removing water from the aqueous formulation comprising the mixture of Vitamin E TPGS and the quantity of a compound or fungal extract of interest to create a solubilizable dry powder.

In another preferred embodiment, the invention includes novel methods of generating a Vitamin E TPGS nanoencapsulated compound in the form of a solubilizable dry powder. In one embodiment, the method may include one or more of the following steps:
sonicating the aqueous formulation comprising a mixture of Vitamin E TPGS and a quantity of a compound or fungal extract of interest to create a plurality of micelles;
freezing the aqueous formulation comprising the mixture of Vitamin E TPGS and the quantity of a compound or fungal extract of interest;
lowering pressure of the aqueous formulation comprising the mixture of Vitamin E TPGS and the quantity of a compound or fungal extract of interest; and
removing water from the aqueous formulation comprising the mixture of Vitamin E TPGS and the quantity of a compound or fungal extract of interest to create a solubilizable dry powder.

As used herein, the term “sonicating” refers to applying sound energy. The chemical effects of sonic waves on chemical systems is called sonochemistry. Sonicating can be used for a variety of purposes, including, but is not limited to, producing nanoparticles, speeding dissolution, and/or disrupting biological material. Many variables, including the power, speed, and ratio of ingredients, can affect the properties of the resulting product. In one embodiment, the power of the sound energy applied can determine the size of micelles and/or reverse micelles.

As used herein, the term “lowering the pressure” refers decreasing the force acting on a unit of area or increasing the area a force is acting on. In one embodiment, pressure is defined as force per unit area. In one embodiment, lowering pressure comprises keeping the force constant while increasing the area. In one embodiment, lowering the pressure comprises keeping the area constant while the force decreases. In one embodiment, pressure is expressed in pascals (Pa). In one embodiment, pressure is expressed in torres (Torr). In one embodiment, pressure is expressed in barye (Ba). In one embodiment, pressure is expressed in standard atmospheres (atm). In some contexts, the word pressure refers to the vapor or equilibrium vapor pressure. Vapor pressure is the pressure exerted by vapor in thermodynamic equilibrium with its condensed phases, either solid or liquid, at a given temperature in a closed system.

As used herein, the term “removing water” refers to eliminating water from a composition such that the composition is substantially free from water. In one embodiment, the composition is 90% free from water. In one embodiment, the composition is 95% free from water. In one embodiment, the composition is 99% free from water. In one embodiment, removing water comprises heating the aqueous formulation. In one embodiment, removing water comprises drying the aqueous formulation for example, by applying a material that absorbs. In one embodiment, removing water comprises applying a vacuum to the aqueous formulation. In one embodiment, removing water comprises suctioning the aqueous formulation. In one embodiment, removing water comprises exposing the aqueous formulation to a desiccant.

In one embodiment, the method disclosed herein comprises adding water to the mixture of Vitamin E TPGS and a quantity of a compound or fungal extract of interest to create an aqueous formulation comprising Vitamin E TPGS and between 1 to 50 mg of a quantity of a compound or fungal extract of interest per mL of water. In another embodiment, the method disclosed herein comprises adding water to the mixture of Vitamin E TPGS and a quantity of a compound or fungal extract of interest to create an aqueous formulation comprising Vitamin E TPGS and greater than mg of a quantity of a compound or fungal extract of interest per mL of water. In another embodiment, the method disclosed herein comprises adding water to the mixture of Vitamin E TPGS and a quantity of a compound or fungal extract of interest to create an aqueous formulation comprising Vitamin E TPGS and less than 50 mg of a quantity of a compound or fungal extract of interest per mL of water. In another embodiment, the method disclosed herein comprises adding water to the mixture of Vitamin E TPGS and a quantity of a compound or fungal extract of interest to create an aqueous formulation comprising Vitamin E TPGS and less than 1 mg of a quantity of a compound or fungal extract of interest per mL of water. In another embodiment, the method disclosed herein comprises adding water to the mixture of Vitamin E TPGS and a quantity of a compound or fungal extract of interest to create an aqueous formulation comprising Vitamin E TPGS and less than 0.01 mg of a quantity of a compound or fungal extract of interest per mL of water.

As used herein, the term “compound” or “one or more compounds” means that at least one compound derived from a fungal extract, or a synthetic or semi-synthetic version of the same. As known in the art, the term “compound” does not mean a single molecule, but multiples or moles of one or more compound. As known in the art, the term “compound” means a specific chemical constituent possessing distinct chemical and physical properties, whereas “compounds” refer to one or more chemical constituents. In a preferred embodiment, one or more compounds of the invention may be formulated with TPGS to form a novel nanoemulsion and/or nanoencapsulated complex compositions having preferably, one or more compounds selected from the group consisting of: ganodermic acids, cordycepin, hericenones, erinacines, psilocybin, psilocin, baeocystin, norbaeocystin, psilacetin, and tryptamines or a combination of the same.

As used herein, “ganodermic acids,” (“GMAS”) refer to a class of bioactive compounds closely related triterpenoids derived from Ganoderma lucidum. Exemplary ganodermic acids of the present invention may include one or more of the following:

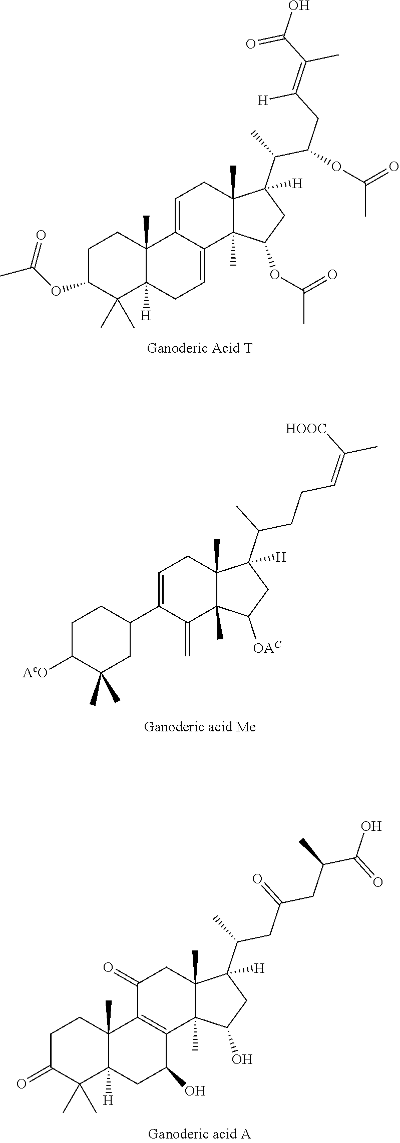

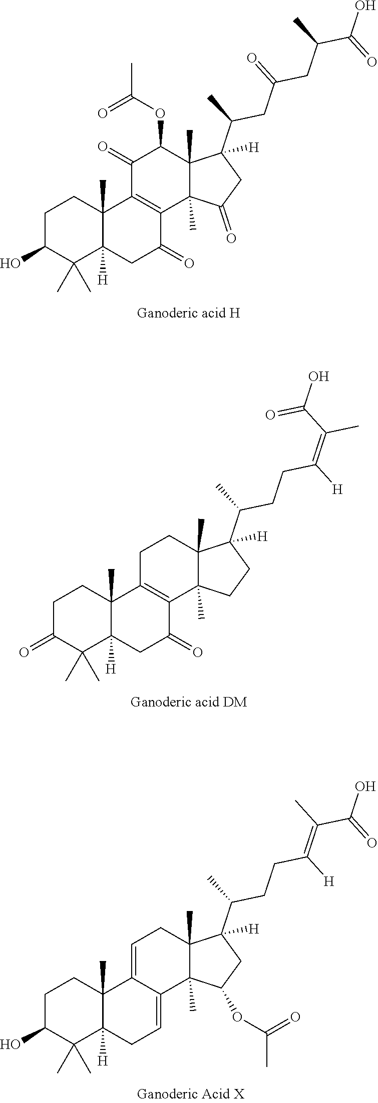 [SMILES: CC(=O)O[C@@H]1C(=O)C2=C(C(=O)C[C@H]3C(C)(C)[C@@H](O)CC[C@]23C)[C@]2(C)C(=O)C[C@H]([C@@H](C)CC(=O)C[C@@H](C)C(=O)O)[C@@]12C ; C/C(=C/CC[C@@H](C)[C@H]1CC[C@@]2(C)C3=C(CC[C@]12C)[C@@]1(C)CCC(=O)C(C)(C)[C@@H]1CC3=O)C(=O)O ; CC(=O)O[C@H]1C[C@H]([C@H](C)CC/C=C(\C)C(=O)O)[C@@]2(C)CC=C3C(=CC[C@H]4C(C)(C)[C@@H](O)CC[C@]34C)[C@]12C]

Within the context of this disclosure, compositions comprising one or more GMAS are formulated with a solubilizing excipient, which in a preferred embodiment may include TPGS, forming a novel nanoencapsulated complex containing one or more GMAS thereby providing novel aqueous formulations having enhanced solubility and bioavailability. In one embodiment, the compositions disclosed herein comprise a ratio of Vitamin E TPGS to the first purified compound of about 90:10 to about 70:30 by percent mass. In one embodiment, the purified compound is contained within a micelle of Vitamin E TPGS.

As used herein, “cordycepin,” also referred to as 3′-deoxyadenosine, refers generally to a bioactive compound derived from Cordyceps militaris, and is a derivative of the nucleoside adenosine, differing from the latter by the absence of the hydroxy group in the 3′ position of its ribose moiety.

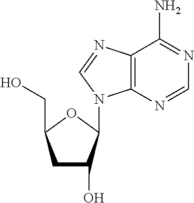

Within the context of this disclosure, compositions comprising cordycepin is formulated with a solubilizing excipient, which in a preferred embodiment may include TPGS, forming a novel nanoencapsulated complex containing cordycepin thereby providing a novel aqueous formulation having enhanced solubility and bioavailability.

As used herein, “hericenones,” refer to a class of bioactive compounds derived from Hericium erinaceum. (also referred to herein as Lion's Mane). Hericenones are low molecular weight and hydrophobic compounds that have shown the ability to stimulate nerve growth factor (NGF). Example compositions of this class include:

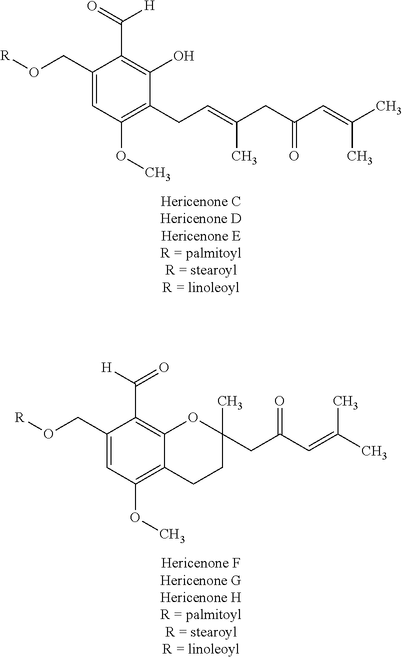

Within the context of this disclosure, compositions comprising one or more hericenones are formulated with a solubilizing excipient, which in a preferred embodiment may include TPGS, forming a novel nanoencapsulated complex containing one or more hericenones thereby providing novel aqueous formulations having enhanced solubility and bioavailability.

As used herein, “erinacines,” refer to a class of bioactive compounds derived from Hericium erinaceum and belong to the group of cyathin diterpenoids that show biological activities as stimulators of NGF. Example compositions of this class include:

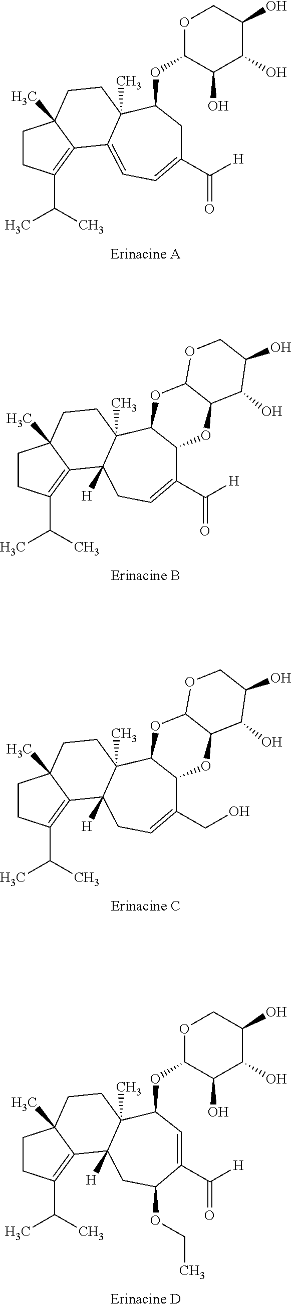

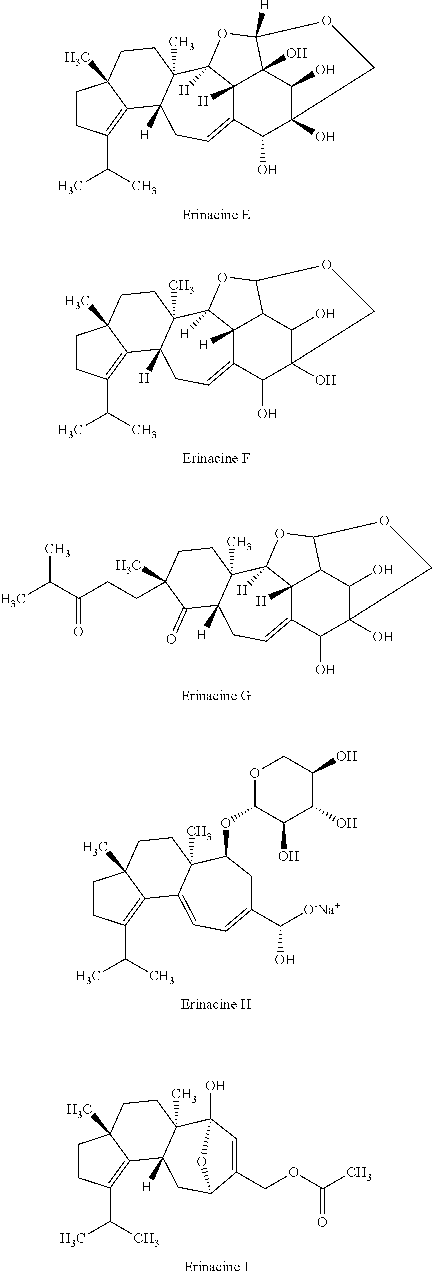

Within the context of this disclosure, compositions comprising one or more erinacines are formulated with a solubilizing excipient, which in a preferred embodiment may include TPGS, forming a novel nanoencapsulated complex containing one or more erinacines thereby providing novel aqueous formulations having enhanced solubility and bioavailability.

As used herein, “psilocybin,” refers to the bioactive compound [3-(2-dimethylaminoethyl)-1H-indol-4-yl] dihydrogen phosphate having the following chemical formula:

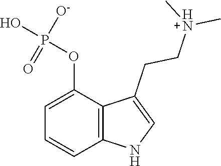

In one embodiment, psilocybin or psilocin may be derived from a “psilocybin mushroom,” which includes a polyphyletic, informal group of fungi that contain psilocybin, psilocin or both within their biomass, typically within their fruiting bodies, resulting in their activation of a psychedelic reaction in a subject. Biological genera containing psilocybin mushrooms within the scope of the invention include: Copelandia, Gymnopilus, Inocybe, Panaeolus, Pholiotina, Pluteus, and Psilocybe. Within the context of this disclosure, compositions comprising psilocybin is formulated with a solubilizing excipient, which in a preferred embodiment may include TPGS, forming a novel nanoencapsulated complex containing psilocybin thereby providing a novel pharmaceutical formulations having enhanced solubility and bioavailability.

As used herein, “psilocin,” refer to the bioactive compound that is the metabolized version of psilocybin, and is also known as 4-hydroxy-N,N-dimethyltryptamine having the following chemical formula:

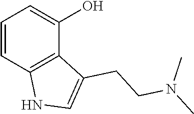

Within the context of this disclosure, compositions comprising psilocin is formulated with a solubilizing excipient, which in a preferred embodiment may include TPGS, forming a novel nanoencapsulated complex containing psilocin thereby providing a novel aqueous formulation having enhanced solubility and bioavailability.

As used herein, “baeocystin,” refers to an alkaloid and analog of psilocybin. It is found as a minor compound in most psilocybin mushrooms together with psilocybin, norbaeocystin, and psilocin. Baeocystin is an N-demethylated derivative of psilocybin, and a phosphorylated derivative of 4-HO-NMT (4-hydroxy-N-methyltryptamine) having the following chemical formula:

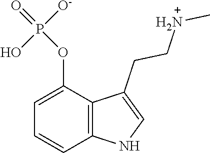 [SMILES: C[NH2+]CCc1c[nH]c2cccc(OP(=O)([O-])O)c12]

Within the context of this disclosure, compositions comprising baeocystin is formulated with a solubilizing excipient, which in a preferred embodiment may include TPGS, forming a novel nanoencapsulated complex containing baeocystin thereby providing a novel aqueous formulation having enhanced solubility and bioavailability.

As used herein, “norbeocisitin,” refers to refers to an alkaloid and analog of psilocybin. It is found as a minor compound in most psilocybin mushrooms together with psilocybin, baeocystin, and psilocin. Norbeocisitin is a N-demethylated derivative of baeocystin (itself a N-demethylated derivative of psilocybin), and a phosphorylated derivative of 4-hydroxytryptamine having the following chemical formula:

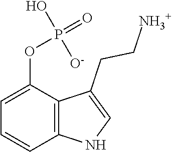 [SMILES: [NH3+]CCc1c[nH]c2cccc(OP(=O)([O-])O)c12]

Within the context of this disclosure, compositions comprising norbeocisitin is formulated with a solubilizing excipient, which in a preferred embodiment may include TPGS, forming a novel nanoencapsulated complex containing norbeocisitin thereby providing a novel aqueous formulation having enhanced solubility and bioavailability.

As used herein, “psilacetin,” also known as “O-Acetylpsilocin” refers to a is a semi-synthetic pro-drug analog of psilocybin having the following chemical formula:

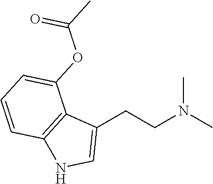 [SMILES: CC(=O)Oc1cccc2[nH]cc(CCN(C)C)c12]

Within the context of this disclosure, compositions comprising psilacetin is formulated with a solubilizing excipient, which in a preferred embodiment may include TPGS, forming a novel nanoencapsulated complex containing psilacetin thereby providing a novel aqueous formulation having enhanced solubility and bioavailability.

As used herein, “tryptamines” or “natural tryptamines,” refers to the class of a monoamine alkaloids containing an indole ring structure that is structurally similar to the amino acid tryptophan having the following generalized formula:

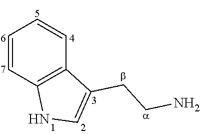 [SMILES: NCCc1c[nH]c2ccccc12]

Within the context of this disclosure, compositions comprising one or more tryptamines are formulated with a solubilizing excipient, which in a preferred embodiment may include TPGS, forming a novel nanoencapsulated complex containing one or more tryptamines thereby providing novel aqueous formulations having enhanced solubility and bioavailability.

As used herein, “reishi extract,” or “reishi,” refers to the class of a compounds, such as polysaccharides, triterpenes, and fatty acids derived from the fungus Ganoderma lucidum. Within the context of this disclosure, compositions comprising a reishi extract are formulated with a solubilizing excipient, which in a preferred embodiment may include TPGS, forming a novel nanoencapsulated complex containing a reishi extract thereby providing novel aqueous formulations having enhanced solubility and bioavailability.

The composition of the invention may include a variety of solubilizing surfactants, which may preferably be configured to solubilize one or more fungal compounds of interest, for example through the formation of a nanoemulsion and/or nanoencapsulated complexes containing one or more said fungal compounds of interest. The surfactants for use in connection with the present invention include, but are not limited to, sorbitan fatty acid esters (e.g., Spans®), polyoxyethylene sorbitan fatty acid esters (e.g., Tweens®), sodium lauryl sulfate (SLS), sodium dodecylbenzene sulfonate (SDBS) dioctyl sodium sulfosuccinate (Docusate), dioxycholic acid sodium salt (DOSS), Sorbitan Monostearate, Sorbitan Tristearate, hexadecyltrimethyl ammonium bromide (HTAB), Sodium N-lauroylsarcosine, Sodium Oleate, Sodium Myristate, Sodium Stearate, Sodium Palmitate, Gelucire 44/14, ethyl enediamine tetraacetic acid (EDTA), Vitamin E d-alpha tocopheryl polyethylene glycol 1000 succinate (TPGS), Lecithin, M W 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, Pluronic F68, Pluronic F108, and Pluronic F 127 (or any other polyoxyethylene-polyoxypropylene co-polymers (Pluronics®) or saturated polyglycolized glycerides (Gelucirs®)). Specific example of such surfactants that may be used in connection with this invention include, but are not limited to, Span 65, Span 25, Tween 20, Capryol 90, Pluronic F108, sodium lauryl sulfate (SLS), Vitamin E TPGS, pluronics and copolymers. SLS is generally preferred.

The amount of the surfactant relative to the total weight of the composition may be between 0.1-15%. Preferably, it is from about 0.5% to about 10%, more preferably from about 0.5 to about 5%, e.g., about 0.5 to 4%, about 0.5 to 3%, about 0.5 to 2%, about 0.5 to 1%, or about 0.5%. In certain embodiments, the amount of the surfactant relative to the total weight of the composition is at least about 0.1%, preferably about 0.5%. In these embodiments, the surfactant would be present in an amount of no more than about 15%, and preferably no more than about 12%, about 11%, about 10%, about 9%, about 8%, about 7%), about 6%), about 5%, about 4%, about 3%, about 2% or about 1%. An embodiment wherein the surfactant is in an amount of about 0.5% by weight is preferred.

The surfactant can have a hydrophilic/lipophilic balance (HLB) value between about 1 and about 25 and a melting point between about 25° C. and about 70° C. The HLB characteristic of surfactants can be determined in accordance with “Physical Pharmacy: Physical Chemical Principles in the Pharmaceutical Sciences,” Fourth Edition, pp. 371-373, A. Martin, Ed., Lippincott Williams & Wilkins, Philadelphia (1993). Preferably, the surfactant is selected from the group consisting of: polyoxyethylene alkyl ethers, polyoxyethylene stearates, polyethylene glycols (PEG), poloxamers, poloxamines, sarcosine based surfactants, polysorbates, aliphatic alcohols, alkyl and aryl sulfates, alkyl and aryl polyether sulfonates and other sulfate surfactants, trimethyl ammonium based surfactants, lecithin and other phospholipids, bile salts, polyoxyethylene castor oil derivatives, polyoxyethylene sorbitan fatty acid esters, Sorbitan fatty acid esters, Sucrose fatty acid esters, alkyl glucopyranosides, alkyl maltopyranosides, glycerol fatty acid esters, Alkyl Benzene Sulphonic Acids, Alkyl Ether Carboxylic Acids, Alkyl and aryl Phosphate esters, Alkyl and aryl Sulphate esters, Alkyl and aryl Sulphonic acids, Alkyl Phenol Phosphates esters, Alkyl Phenol Sulphates esters, Alkyl and Aryl Phosphates, Alkyl Polysaccharides, Alkylamine Ethoxylates, Alkyl-Naphthalene Sulphonates formaldehyde condensates, Sulfosuccinates, lignosulfonates, Ceto-Oleyl Alcohol Ethoxylates, Condensed Naphthalene Sulphonates, Dialkyl and Alkyl Naphthalene Sulphonates, Di-alkyl Sulphosuccinates, Ethoxylated nonylphenols, Ethylene Glycol Esters, Fatty Alcohol Alkoxylates, Hydrogenated tallowalkylamines, Mono-alkyl Sulphosuccinamates, Nonyl Phenol Ethoxylates, Sodium Oleyl N-methyl Taurate, Tallowalkylamines, linear and branched dodecylbenzene sulfonic acids. Preferably, the surfactant is selected from the group consisting of: sodium lauryl sulfate, sodium stearyl sulfate, sodium cetyl sulfate, sodium cetostearyl sulfate, sodium docusate, sodium deoxycholate, N-lauroylsarcosine sodium salt, glyceryl monostearate, glycerol distearate glyceryl palmitostearate, glyceryl behenate, glyceryl caprylate, glyceryl oleate, benzalkonium chloride, CTAB, CTAC, Cetrimide, cetylpyridinium chloride, cetylpyridinium bromide, benzethonium chloride, PEG 40 stearate, PEG 100 stearate, poloxamer 188, poloxamer 338, poloxamer 407 polyoxyl 2 stearyl ether, polyoxyl 100 stearyl ether, polyoxyl 20 stearyl ether, polyoxyl 10 stearyl ether, polyoxyl 20 cetyl ether, polysorbate 20, polysorbate 40, polysorbate 60, polysorbate 61, polysorbate 65, polysorbate 80, polyoxyl 35 castor oil, polyoxyl 40 castor oil, polyoxyl 60 castor oil, polyoxyl 100 castor oil, polyoxyl 200 castor oil, polyoxyl 40 hydrogenated castor oil, polyoxyl 60 hydrogenated castor oil, polyoxyl 100 hydrogenated castor oil, polyoxyl 200 hydrogenated castor oil, cetostearyl alcohol, macrogel 15 hydroxystearate, sorbitan monopalmitate, sorbitan monostearate, sorbitan trioleate, Sucrose Palmitate, Sucrose Stearate, Sucrose Distearate, Sucrose laurate, Glycocholic acid, sodium Glycholate, Cholic Acid, Soidum Cholate, Sodium Deoxycholate, Deoxycholic acid, Sodium taurocholate, taurocholic acid, Sodium taurodeoxy cholate, taurodeoxycholic acid, soy lecithin, phosphatidylcholine, phosphatidylethanolamine, phosphatidylserine, phosphatidylinositol, PEG4000, PEG6000, PEG8000, PEG10000, PEG20000, alkyl naphthalene sulfonate condensate/Lignosulfonate blend, Calcium Dodecylbenzene Sulfonate, Sodium Dodecylbenzene Sulfonate, Diisopropyl naphthaenesulphonate, erythritol distearate, Naphthalene Sulfonate Formaldehyde Condensate, nonylphenol ethoxylate (poe-30), Tristyrylphenol Ethoxylate, Polyoxyethylene (15) tallowalkylamines, sodium alkyl naphthalene sulfonate, sodium alkyl naphthalene sulfonate condensate, sodium alkylbenzene sulfonate, sodium isopropyl naphthalene sulfonate, Sodium Methyl Naphthalene Formaldehyde Sulfonate, sodium n-butyl naphthalene sulfonate, tridecyl alcohol ethoxylate (poe-18), Triethanolamine isodecanol phosphate ester, Triethanolamine tristyrylphosphate ester, Tristyrylphenol Ethoxylate Sulfate, Bis(2-hydroxyethyl)tallowalkylamines. The surfactant system may further include any surfactant as long as it is compatible with pharmaceutical applications.

As detailed below, in some embodiments, the compositions of the present invention contain one or more additional desirable components or compounds. Examples include, but are not limited to, additional active pharmaceutical ingredients as well as excipients, diluents, and carriers such as fillers and extenders (e.g., starch, sugars, mannitol, and silicic derivatives); binding agents {e.g., carboxymethyl cellulose and other cellulose derivatives, alginates, gelatin, and polyvinyl-pyrrolidone); moisturizing agents (e.g., glycerol); disintegrating agents (e.g., calcium carbonate and sodium bicarbonate); agents for retarding dissolution (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; antioxidants; buffers; bacteriostats; coloring agents; perfuming agents; flavoring agents; lubricants (e.g., talc, calcium and magnesium stearate); solid polyethyl glycols; and mixtures thereof. Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, and the like, which are suitable for use in the compositions. It should be understood that the ingredients particularly mentioned above are merely examples and that some embodiments of formulations comprising the compositions of the present invention include other suitable components and agents.

For example, the inventive technology may further include novel a nanoencapsulated complex containing one or more compounds of interest derived from a fungus and/or a fungal extract, and preferably a Vitamin E TPGS nanoencapsulated complex containing one or more compounds of interest derived from a fungus and/or a fungal extract. In one preferred embodiment, the invention may include a pharmaceutical composition as active ingredient an effective amount or dose of one or more of the nanoencapsulated complexes containing one or more compounds of interest derived from a fungus and/or a fungal extract. In some instances, the active ingredient may be provided together with pharmaceutically tolerable adjuvants and/or excipients in the pharmaceutical composition. Such pharmaceutical composition may optionally be in combination with one or more further active ingredients. In one embodiment, one of the aforementioned the novel nanoencapsulated complexes of the invention may act as a drug delivery and dosing system wherein a therapeutically effective amount, or effective dose, or dose may be delivered to a subject in need thereof.

“Pharmaceutical compositions” are compositions that include an amount (for example, a unit dosage) of one or more of the disclosed compounds together with one or more non-toxic pharmaceutically acceptable carriers, including additives, diluents, and/or adjuvants, and optionally other biologically active ingredients. Such pharmaceutical compositions can be prepared by standard pharmaceutical The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The term “therapeutically effective amount” as used herein refers to a dose of a compound, nanoemulsion complex containing one or more active compounds of interest, required to confer a beneficial effect in a subject with a condition or disease in which treatment is sought. A therapeutically efficient amount may vary, as recognized to persons skilled in the art, for instance depending on the route and time of administration, bioavailability of the solubilized nanoemulsion complex compared to a non-treated compound or fungal extract, and physiological characteristics including age, gender and general health of the patient to be treated.

Notably, the terms “therapeutically effective amount” or “effective dose” or “dose” are interchangeably used herein and denote an amount of the pharmaceutical compound having a prophylactically or therapeutically relevant effect on a disease or pathological conditions, i.e., which causes in a tissue, system, animal or human a biological or medical response which is sought or desired, for example, by a researcher or physician. Pharmaceutical formulations can be administered in the form of dosage units which comprise a predetermined amount of active nanoencapsulated compound per dosage unit. The concentration of the prophylactically or therapeutically active ingredient in the formulation may vary from about 0.1 to 100 wt %. Preferably, a nanoencapsulated complex of the invention or the pharmaceutically acceptable salts of the compound of interest contained within a nanoemulsion complex, thereof are administered in doses of approximately 0.5 to 1000 mg, more preferably between 0.1 mg and 1000 mg, 1 and 700 mg, and most preferably 5 and 100 mg per dose unit. Generally, such a dose range is appropriate for total daily incorporation. In other terms, the daily dose is preferably between approximately 0.02 and 100 mg/kg of body weight. The specific dose for each patient depends, however, on a wide variety of factors as already described in the present specification (e.g., depending on the condition treated, the method of administration and the age, weight, and condition of the patient). Preferred dosage unit formulations are those which comprise a daily dose or part-dose, as indicated above, or a corresponding fraction thereof of an active ingredient. Furthermore, pharmaceutical formulations of this type can be prepared using processes generally known in the pharmaceutical art.

The “therapeutically effective amount” for the treatment of a subject afflicted with a disorder ameliorated by the described therapy is an amount that causes amelioration of the disorder being treated or protects against a risk associated with the disorder, and in particular a serotonin receptor related disease or condition. For example, for schizophrenia, a therapeutically effective amount is an amount which causes a significant reduction in psychopathology as determined by clinical improvement; for depression, a therapeutically effective amount is an amount that leads to stabilization and remission of symptoms as measured by the Patient Health Questonnaire-9; for OCD, a therapeutically effective amount is an amount that leads to stabilization and remission of symptoms as measured by the Yale-Brown Obsessive Compulsive Scale; for ADHD, a therapeutically effective amount is an amount that leads to stabilization and remission of symptoms as measured by either the ADHD Rating Scale V or ADHD Self-Report Scale; for eating disorders, a therapeutically effective amount is an amount that leads to stabilization and remission of symptoms as measured by the Eating Disorder Examination Questionnaire; for autism spectrum disorders a therapeutically effective amount is an amount that leads to stabilization and remission of symptoms as measured by physicians' assessment; for PTSD a therapeutically effective amount is an amount that leads to stabilization and remission of symptoms as measured by the Clinician-Administered PT SD Scale for DSM-5; for anxiety, a therapeutically effective amount is an amount that leads to stabilization and remission of symptoms as measured by the General Anxiety Disorder-7; for addiction, a therapeutically effective amount is an amount that leads to stabilization and remission of symptoms as measured by physicians' assessment; for cluster headaches, a therapeutically effective amount is an amount that leads to stabilization and remission of symptoms as measured by the Cluster Headache Severity Scale (CHSS); for dementia, a therapeutically effective amount is an amount that leads to stabilization and remission of symptoms as measured by the Dementia Rating Scale (DRS); for Alzheimer's disease, a therapeutically effective amount is an amount that leads to stabilization and remission of symptoms as measured by the Alzheimer's Disease Assessment Scale-Cognitive Subscale (ADAS-Cog); for paralysis, a therapeutically effective amount is an amount that leads to stabilization and remission of symptoms as measured by physicians' assessment.

As used herein, “treating” or “treatment” describes the management and care of a patient for the purpose of combating a disease, condition, or disorder, and in particular serotonin receptor related disease or condition and includes the administration of a compound or pharmaceutical compositions of the present invention to prevent the onset of the symptoms or complications, alleviating the symptoms or complications, or eliminating the disease, condition or disorder. More specifically, “treating” includes reversing, attenuating, alleviating, minimizing, suppressing or halting at least one deleterious symptom or effect of a disease (disorder) state, disease progression, or other abnormal condition, and in particular a serotonin receptor related disease or condition. Treatment is continued as long as symptoms and/or pathology ameliorate. The term “beneficial” or “improved outcomes” as used herein in the context of treating a condition, refers to extended relieve of symptoms (duration) and/or a more significant reduction of symptoms (magnitude), and in particular with respect to a serotonin receptor related disease or disorder. Exemplary serotonin receptor related diseases or disorders can include schizophrenia, depression, obsessive compulsive disorder, attention deficit-hyperactivity disorders, eating disorders, autism spectrum disorders, PTSD and anxiety as discussed above. The phrase “improved outcomes” means an extended relieve of symptoms (duration) and/or a more significant reduction of symptoms (magnitude).

As used herein, a “serotonin receptor related disease or condition” includes a disease or condition in a subject, and preferably a human subject for which administration of a serotonin receptor is beneficial, or for which modulation of the serotonin receptor's activity results in improved outcomes in the subject.

As noted above, the present invention allows the scaled production of solubilized compounds of interest derived from a fungus and/or a fungal extract. Because of this increased solubility, the invention allows for a variety of compositions having one or more bioactive compounds of interest to be maintained in an aqueous suspension. As a result, the present invention may allow for the production of a variety of compositions for the food and beverage industry, as well as pharmaceutical applications wherein the increased solubility, bioavailability, and other enhanced characteristics, such as more rapid onset time may be commercially and therapeutically beneficial.

In one embodiment, the invention may include aqueous compositions containing one or more nanoencapsulated complexes (the term being generally used to describe a compound of interest derived from a fungus and/or a fungal extract contained within a solubilizing nanoencapsulated complex, and preferably a compound of interest derived from a fungus and/or a fungal extract contained within a Vitamin E TPGS nanoencapsulated complex, that may be introduced to a food or beverage. In a preferred embodiment, the invention may include an aqueous solution containing one or more solubilizing nanoencapsulated complexes. Here, the solubilizing nanoencapsulated complex may be generated as generally described herein. Moreover, in this embodiment, the aqueous solution may contain one or more of the following additional components: saline, purified water, propylene glycol, deionized water, and/or an alcohol such as ethanol, as well as a pH buffer that may allow the aqueous solution to be maintained at a desired. Additional embodiments may include the addition of an acid or base that may adjust the solutions pH, such as formic acid, or ammonium hydroxide.

In another embodiment, the invention may include a consumable food additive having at least one nanoencapsulated complex, and preferably a Vitamin E TPGS nanoencapsulated complex. This consumable food additive may further include one or more food additive polysaccharides, such as dextrin and/or maltodextrin, as well as an emulsifier. Example emulsifiers may include, but not be limited to: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.

The consumable food additive of the invention may be a homogenous composition and may further comprise a flavoring agent. Exemplary flavoring agents may include sucrose (sugar), glucose, fructose, sorbitol, mannitol, corn syrup, high fructose corn syrup, saccharin, aspartame, sucralose, acesulfame potassium (acesulfame-K), neotame. The consumable food additive of the invention may also contain one or more coloring agents. Exemplary coloring agents may include: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate. In one embodiment, this a nanoencapsulated complex, and preferably a Vitamin E TPGS nanoencapsulated complex may be lyophilized forming a water-soluble powder. In certain preferred embodiments, one or more flavoring agents may be added to a quantity of powdered or lyophilized nanoencapsulated complex compositions.

The consumable food additive of the invention may also contain one or more surfactants, such as glycerol monostearate and polysorbate 80. The consumable food additive of the invention may also contain one or more preservatives. Exemplary preservatives may include ascorbic acid, citric acid, sodium benzoate, calcium propionate, sodium erythorbate, sodium nitrite, calcium sorbate, potassium sorbate, BHA, BHT, EDTA, or tocopherols. The consumable food additive of the invention may also contain one or more nutrient supplements, such as: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.

In one embodiment, the invention may include a consumable fluid containing at least one nanoencapsulated complex, and preferably a nanoencapsulated complex derived from Vitamin E TPGS. In one preferred embodiment, this consumable fluid may be added to a drink or beverage to infuse it with the nanoencapsulated complex generated as generally herein described. The consumable fluid may include a food additive polysaccharide such as maltodextrin and/or dextrin, which may further be in an aqueous form and/or solution. For example, in one embodiment, an aqueous maltodextrin solution may include a quantity of sorbic acid and an acidifying agent to provide a food grade aqueous solution of maltodextrin having a pH of 2-4 and a sorbic acid content of 0.02-0.1% by weight.

In certain embodiments, the consumable fluid may include water, as well as an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water. The consumable fluid may further include at least one additional ingredient, including but not limited to: xanthan gum, cellulose gum, whey protein hydrolysate, ascorbic acid, citric acid, malic acid, sodium benzoate, sodium citrate, sugar, phosphoric acid, and water. In certain embodiments, the consumable fluid of the invention may be generated by addition of a quantity of nanoencapsulated complex in powder or liquid form as generally described herein to an existing consumable fluid, such as a branded beverage or drink.

In one embodiment, the invention may include a consumable gel having at least one nanoencapsulated complex, and preferably a nanoencapsulated complex derived from Vitamin E TPGS, and gelatin in an aqueous solution. Additional embodiments may include a liquid composition having at least one nanoencapsulated complex, and preferably a nanoencapsulated complex derived from Vitamin E TPGS, in a first quantity of water; and at least one of: xanthan gum, cellulose gum, whey protein hydrolysate, ascorbic acid, citric acid, malic acid, sodium benzoate, sodium citrate, sugar, phosphoric acid, and/or a sugar alcohol. In one preferred embodiment, the composition may further include a quantity of ethanol. Here, the amount of nanoencapsulated complex may include: less than 10 mass % water; more than 95 mass % water; about 0.1 mg to about 1000 mg of the nanoencapsulated complex; about 0.1 mg to about 500 mg of the nanoencapsulated complex; about 0.1 mg to about 200 mg of the nanoencapsulated complex; about 0.1 mg to about 100 mg of the nanoencapsulated complex; about 0.1 mg to about 100 mg of the nanoencapsulated complex; about 0.1 mg to about 10 mg of the nanoencapsulated complex; about 0.5 mg to about 5 mg of the nanoencapsulated complex; about 1 mg/kg to 5 mg/kg (body weight) in a human of the nanoencapsulated complex. In alternative embodiments, the composition may include at least one nanoencapsulated complex, in the range of 50 mg/L to 300 mg/L; at least one nanoencapsulated complex in the range of 50 mg/L to 100 mg/L; at least one nanoencapsulated complex in the range of 50 mg/L to 500 mg/L; at least one nanoencapsulated complex over 500 mg/L; at least one nanoencapsulated complex under 50 mg/L. Additional embodiments may include one or more of the following additional components: a flavoring agent; a coloring agent; and/or caffeine.

In one embodiment, the invention may include a liquid composition having at least one nanoencapsulated complex, and preferably a nanoencapsulated complex derived from Vitamin E TPGS, being solubilized in said first quantity of water and a first quantity of ethanol in a liquid state. In a preferred embodiment, a first quantity of ethanol in a liquid state may be between 1% to 20% or more weight by volume of the liquid composition. Such nanoencapsulated complexes may be generated as herein identified. In a preferred embodiment, the ethanol or ethyl alcohol component may be up to about ninety-nine-point nine five percent (99.95%) by weight and the nanoencapsulated complex about zero-point zero five percent (0.05%) by weight. Examples of the preferred embodiment may include liquid ethyl alcohol compositions at least one nanoencapsulated complex, and preferably a nanoencapsulated complex derived from Vitamin E TPGS, wherein said ethyl alcohol has a proof greater than 100, and/or less than 100. Additional examples of a liquid composition containing ethyl alcohol and at least one nanoencapsulated complex, and preferably a nanoencapsulated complex derived from Vitamin E TPGS, may include, beer, wine, and/or distilled spirits.

Additional embodiments of the invention may include a chewing gum composition having a first quantity of at least one nanoencapsulated complex, and preferably a nanoencapsulated complex derived from Vitamin E TPGS. In a preferred embodiment, a chewing gum composition may further include a gum base comprising a buffering agent selected from the group consisting of acetates, glycinates, phosphates, carbonates, glycerophosphates, citrates, borates, and mixtures thereof. Additional components may include at least one sweetening agent and at least one flavoring agent.

In one embodiment, the chewing gum composition described above may include:
0.01 to 1% by weight of at least one nanoencapsulated complex;
25 to 85% by weight of a gum base;
10 to 35% by weight of at least one sweetening agent; and
1 to 10% by weight of a flavoring agent.

Here, such flavoring agents may include menthol flavor, eucalyptus, cinnamon, mint flavor and/or L-menthol. Sweetening agents may include one or more of the following: xylitol, sorbitol, isomalt, aspartame, sucralose, acesulfame potassium, and saccharin. Additional preferred embodiment may include a chewing gum having a pharmaceutically acceptable excipient selected from the group consisting of fillers, disintegrants, binders, lubricants, and antioxidants. The chewing gum composition may further be non-disintegrating and also include one or more coloring and/or flavoring agents.

The invention may further include a composition for a nanoencapsulated complex infused solution comprising essentially of water and/or purified water, at least one nanoencapsulated complex, and preferably a nanoencapsulated complex derived from Vitamin E TPGS, and at least one flavoring agent. A nanoencapsulated complex infused solution of the invention may further include a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of the nanoencapsulated complex infused solution may include, but not be limited to sodium chloride, sodium chloride solution, glycerin, a coloring agent, and a demulcent. As to this last potential component, in certain embodiments, a demulcent may include pectin, glycerin, honey, methylcellulose, and/or propylene glycol. As noted above, in a preferred embodiment, a nanoencapsulated complex may include at least one nanoencapsulated complex wherein such nanoencapsulated complexes may be generated as herein described.

The invention may further include a composition for a nanoencapsulated complex infused anesthetic solution having water, or purified water, at least one nanoencapsulated complex, and at least one oral anesthetic. In a preferred embodiment, an anesthetic may include benzocaine, and/or phenol in a quantity of between 0.1% to 15% volume by weight. Additional embodiments may include a nanoencapsulated complex infused anesthetic solution having a sweetener which may be selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of a nanoencapsulated complex infused solution may include, but not be limited to sodium chloride, sodium chloride solution, glycerin, a coloring agent, and a demulcent. In a preferred embodiment, a demulcent may be selected from the group consisting of pectin, glycerin, honey, methylcellulose, and propylene glycol.

The invention may further include a composition for a hard lozenge or candy for rapid delivery of nanoencapsulated complexes, and preferably a nanoencapsulated complex derived from Vitamin E TPGS, through the oral mucosa. In this embodiment, such a hard lozenge composition may include: a crystalized sugar base, and at least one nanoencapsulated complex, wherein the hard lozenge has a moisture content between 0.1 to 2%. In this embodiment, the nanoencapsulated complex may be added to the sugar base when it is in a liquefied form and prior to the evaporation of the majority of water content. Such a hard lozenge may further be referred to as a candy. In a preferred embodiment, a crystalized sugar base may be formed from one or more of the following: sucrose, invert sugar, corn syrup, and isomalt or a combination of the same. Additional components may include at least one acidulant. Examples of acidulants may include, but not be limited to citric acid, tartaric acid, fumaric acid, and malic acid. Additional components may include at least one pH adjustor. Examples of pH adjustors may include, but not be limited to calcium carbonate, sodium bicarbonate, and magnesium trisilicate. In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The hard lozenge composition may also include a demulcent, for example: pectin, glycerin, honey, methylcellulose, propylene glycol, and glycerin. In this embodiment, a demulcent may be in a quantity between 1 mg to 10 mg.

The invention may include a chewable lozenge or candy for rapid delivery of nanoencapsulated complexes through the oral mucosa. In a preferred embodiment, the compositions may include: a glycerinated gelatin base, at least one sweetener, and at least one nanoencapsulated complex dissolved in a first quantity of water. In this embodiment, a sweetener may include a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components may include at least one acidulant. Examples of acidulants may include, but not be limited to citric acid, tartaric acid, fumaric acid, and malic acid. Additional components may include at least one pH adjustor. Examples of pH adjustors may include, but not be limited to calcium carbonate, sodium bicarbonate, and magnesium trisilicate. In another preferred embodiment, the composition may include at least one anesthetic. Example of such anesthetics may include benzocaine and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The chewable lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a nanoencapsulated complex may include at least one nanoencapsulated complex, and preferably a nanoencapsulated complex derived from Vitamin E TPGS.

The invention may include a soft lozenge for rapid delivery of nanoencapsulated complexes through the oral mucosa. In a preferred embodiment, the compositions may include: a polyethylene glycol base, at least one sweetener, and at least one nanoencapsulated complex dissolved in a first quantity of water. In this embodiment, a sweetener may include sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components may include at least one acidulant. Examples of acidulants may include, but not be limited to citric acid, tartaric acid, fumaric acid, and malic acid. Additional components may include at least one pH adjustor. Examples of pH adjustors may include, but not be limited to calcium carbonate, sodium bicarbonate, and magnesium tri silicate.

In another preferred embodiment, the composition may include at least one anesthetic. Example of such anesthetics may include benzocaine and phenol. In this embodiment, a first quantity of anesthetic may be between 1 mg to 15 mg per lozenge. Additional embodiments may include a quantity of menthol. In this embodiment, such a quantity of menthol may be between 1 mg to 20 mg. The soft lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a nanoencapsulated complex may preferably be a nanoencapsulated complex derived from Vitamin E TPGS.

In another embodiment, the invention may include a tablet or capsule consisting essentially of a nanoencapsulated complex and a pharmaceutically acceptable excipient. Examples may include solid, semi-solid, and aqueous excipients such as: maltodextrin, whey protein isolate, xanthan gum, guar gum, diglycerides, monoglycerides, carboxymethyl cellulose, glycerin, gelatin, polyethylene glycol and water-based excipients. In this embodiment, a nanoencapsulated complex may preferably be a nanoencapsulated complex derived from Vitamin E TPGS, and may have an improved shelf-life, composition stability, and bioavailability upon ingestion.

In a preferred embodiment, a tablet or capsule may include an amount of nanoencapsulated complex of 5 milligrams or less. Alternative embodiments may include an amount of nanoencapsulated complex between 5 milligrams and 200 milligrams. Still other embodiments may include a tablet or capsule having an amount of nanoencapsulated complex that is more than 200 milligrams. Still other embodiments may include a tablet or capsule having an amount of nanoencapsulated complex that is more than 500 milligrams. The invention may further include a method of manufacturing and packaging a nanoencapsulated complex dosage, consisting of the following steps: 1) preparing a fill solution with a desired concentration of a nanoencapsulated complexes in a liquid carrier wherein said nanoencapsulated complex is dissolved in said liquid carrier; 2) encapsulating said fill solution in capsules; 3) packaging said capsules in a closed packaging system; and 4) removing atmospheric air from the capsules. In one embodiment, the step of removing atmospheric air consists of purging the packaging system with an inert gas, such as, for example, nitrogen gas, such that said packaging system provides a room temperature stable product. In one preferred embodiment, the packaging system may include a blister package, which may be constructed of material that minimizes exposure to moisture and air.

In one embodiment, a preferred liquid carrier may include a water-based carrier, such as for example an aqueous sodium chloride solution. In one embodiment, a desired nanoencapsulated complex concentration may be about 1-10% w/w, while in other embodiments it may be about 1.5-6.5% w/w. Alternative embodiments may include an amount of nanoencapsulated complex between 5 milligrams and 200 milligrams. Still, other embodiments may include a tablet or capsule having amount of nanoencapsulated complex that is more than 200 milligrams. Other embodiments may include a tablet or capsule having an amount of nanoencapsulated complex that is more than 500 milligrams.

The invention may include an oral pharmaceutical solution, such as a sub-lingual spray having nanoencapsulated complexes, and preferably be a nanoencapsulated complex derived from Vitamin E TPGS and a liquid carrier. One embodiment may include a nanoencapsulated complex, 30-33% w/w water, about 50% w/w alcohol, 0.01% w/w butylated hydroxylanisole (BHA) or 0.1% w/w ethylenediaminetetraacetic acid (EDTA) and 5-21% w/w co-solvent, having a combined total of 100%, wherein said co-solvent is selected from the group consisting of propylene glycol, polyethylene glycol, and combinations thereof. In an alternative embodiment, such an oral pharmaceutical solution may consist essentially of 0.1 to 5% w/w of said nanoencapsulated complex, about 50% w/w alcohol, 5.5% w/w propylene glycol, 12% w/w polyethylene glycol and w/w water. In a preferred composition, the alcohol component may be ethanol.

The invention may include an oral pharmaceutical solution, such as a sublingual spray, consisting essentially of about 0.1% to 1% w/w nanoencapsulated complexes, about 50% w/w alcohol, 5.5% w/w propylene glycol, 12% w/w polyethylene glycol, 30-33% w/w water, 0.01% w/w butylated hydroxyanisole, having a combined total of 100%, and wherein said nanoencapsulated complex is a nanoencapsulated complex derived from Vitamin E TPGS and a liquid carrier. In an alternative embodiment, such an oral pharmaceutical solution may consist essentially of 0.54% w/w nanoencapsulated complex, 31.9% w/w water, 12% w/w polyethylene glycol 400, 5.5% w/w propylene glycol, 0.01% w/w butylated hydroxyanisole, 0.05% w/w sucralose, and 50% w/w alcohol, wherein the a the alcohol components may be ethanol.

The invention may include a solution for nasal and/or sublingual administration of a nanoencapsulated complex including: 1) an excipient of propylene glycol, ethanol anhydrous, or a mixture of both; and 2) a nanoencapsulated complex which may a nanoencapsulated complex derived from Vitamin E TPGS. In a preferred embodiment, the composition may further include a topical decongestant, which may include phenylephrine hydrochloride, Oxymetazoline hydrochloride, and Xylometazoline in certain preferred embodiments. The composition may further include an antihistamine, and/or a steroid. Preferably, the steroid component is a corticosteroid selected from the group consisting of: neclomethasone dipropionate, budesonide, ciclesonide, flunisolide, fluticasone furoate, fluticasone propionate, mometasone, and triamcinolone acetonide. In alternative embodiments, the solution for nasal and/or sublingual administration of a nanoencapsulated complex may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, and propylene glycol.

The invention may further include an aqueous solution for nasal and/or sublingual administration of a nanoencapsulated complex comprising: a water and/or saline solution; and a nanoencapsulated complex, such as a nanoencapsulated complex derived from Vitamin E TPGS, In a preferred embodiment, the composition may further include a topical decongestant, which may include phenylephrine hydrochloride, Oxymetazoline hydrochloride, and Xylometazoline in certain preferred embodiments. The composition may further include an antihistamine and/or a steroid. Preferably, the steroid component is a corticosteroid selected from the group consisting of: neclomethasone dipropionate, budesonide, ciclesonide, flunisolide, fluticasone furoate, fluticasone propionate, mometasone, and triamcinolone acetonide. In alternative embodiments, the aqueous solution may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, or propylene glycol.

The invention may include a topical formulation for the transdermal delivery of nanoencapsulated complexes. In a preferred embodiment, a topical formulation for the transdermal delivery of nanoencapsulated complexes which may include at least one nanoencapsulated complex derived from Vitamin E TPGS, and another pharmaceutically acceptable excipient. Preferably a pharmaceutically acceptable excipient may include one or more: gels, ointments, cataplasms, poultices, pastes, creams, lotions, plasters, and jellies or even polyethylene glycol. Additional embodiments may further include one or more of the following components: a quantity of capsaicin; a quantity of benzocaine; a quantity of lidocaine; a quantity of camphor; a quantity of benzoin resin; a quantity of methylsalicilate; a quantity of triethanolamine salicylate; a quantity of hydrocortisone; or a quantity of salicylic acid.

The invention may include a gel for transdermal administration of a nanoencapsulated complex which may include at least one nanoencapsulated complex derived from Vitamin E TPGS, which may be generated as herein described. In this embodiment, the mixture preferably contains from 15% to about 90% ethanol, about 10% to about 60% buffered aqueous solution or water, about 0.1 to about 25% propylene glycol, from about 0.1 to about 20% of a gelling agent, from about 0.1 to about 20% of a base, from about 0.1 to about 20% of an absorption enhancer and from about 1% to about 25% polyethylene glycol, and a nanoencapsulated complex as generally described herein.

In another embodiment, the invention may further include a transdermal composition having a pharmaceutically effective amount of a nanoencapsulated complex for delivery of the nanoencapsulated complex containing the compound of interest to the bloodstream of a user. This transdermal composition may include a pharmaceutically acceptable excipient and at least one nanoencapsulated complex, which may include at least one nanoencapsulated complex derived from Vitamin E TPGS, and which may be generated as herein described, wherein the nanoencapsulated complex is capable of diffusing from the composition into the bloodstream of the user. In a preferred embodiment, a pharmaceutically acceptable excipient to create a transdermal dosage form selected from the group consisting of gels, ointments, cataplasms, poultices, pastes, creams, lotions, plasters, and jellies. The transdermal composition may further include one or more surfactants. In one preferred embodiment, the surfactant may include a surfactant-lecithin organogel, which may further be present in an amount of between about 95% and about 98% w/w. In an alternative embodiment, a surfactant-lecithin organogel comprises lecithin and PPG-2 myristyl ether propionate and/or high molecular weight polyacrylic acid polymers. The transdermal composition may further include a quantity of isopropyl myristate.

The invention may further include transdermal compositions having one or more permeation enhancers to facilitate transfer of the nanoencapsulated complex across a dermal layer. In a preferred embodiment, a permeation enhancer may include one or more of the following: propylene glycol monolaurate, diethylene glycol monoethyl ether, an oleoyl macrogolglyceride, a caprylocaproyl macrogolglyceride, and an oleyl alcohol.

The invention may also include a liquid nanoencapsulated complex liniment composition consisting of water, isopropyl alcohol solution, and a nanoencapsulated complex, which may include at least one nanoencapsulated complex derived from Vitamin E TPGS, and which may be generated as herein described. This liquid nanoencapsulated complex liniment composition may further include approximately 97.5% to about 99.5% by weight of 70% isopropyl alcohol solution and from about 0.5% to about 2.5% by weight of a nanoencapsulated complex mixture.

Based on the improved solubility and other physical properties, as well as cost advantages, improved nanoencapsulated complex affinity and capacity, extended shelf-life, and scalability of the invention's nanoencapsulated complex production incorporating platform including at least one nanoencapsulated complex derived from Vitamin E TPGS, the invention may include one or more commercial infusions. For example, commercially available products, such a lip balm, soap, shampoos, lotions, creams, and cosmetics may be infused with one or more nanoencapsulated complexes.

In one embodiment, the invention may include one or more methods of treating a medical condition in a mammal. In this embodiment, the novel method may include of administering a therapeutically effective amount of a nanoencapsulated complex, for example, at least one nanoencapsulated complex derived from Vitamin E TPGS, wherein the medical condition or therapeutic result is selected from the group consisting of: a learning disability, mental health conditions, depression, anxiety, OCD, addiction, PTSD, eating disorder, Alzheimer's disease, dementia, nerve damage, stroke, Parkinson's disease, brain damage, decreased cognitive function, paralysis, down syndrome, autism, decreased cognition, neuron regrowth, cardiovascular disease, weakened immune system, viral infection, COVID-19 infection, anti-bacterial infection, and any combination thereof.

In a preferred embodiment, the pharmaceutical composition may be administered by a route selected from the group consisting of transdermal, topical, oral, buccal, sublingual, intra-venous, intra-muscular, vaginal, rectal, ocular, nasal, and follicular. The amount of nanoencapsulated complex may be a therapeutically effective amount, which may be determined by the patient's age, weight, medical condition compound-delivered, route of delivery, and the like. In one embodiment, a therapeutically effective amount may be 50 mg or less of a nanoencapsulated complex. In another embodiment, a therapeutically effective amount may be 50 mg or more of a nanoencapsulated complex. It should be noted that for any of the above composition, unless otherwise stated, an effective amount of nanoencapsulated complexes may include amounts between: 0.01 mg to 0.1 mg; 0.01 mg to 0.5 mg; 0.01 mg to 1 mg; 0.01 mg to 5 mg; 0.01 mg to 10 mg; 0.01 mg to 25 mg; 0.01 mg to 50 mg; 0.01 mg to 75 mg; 0.01 mg to 100 mg; 0.01 mg to 125 mg; 0.01 mg to 150 mg; 0.01 mg to 175 mg; 0.01 mg to 200 mg; 0.01 mg to 225 mg; 0.01 mg to 250 mg; 0.01 mg to 275 mg; 0.01 mg to 300 mg; 0.01 mg to 225 mg; 0.01 mg to 350 mg; 0.01 mg to 375 mg; 0.01 mg to 400 mg; 0.01 mg to 425 mg; 0.01 mg to 450 mg; 0.01 mg to 475 mg; 0.01 mg to 500 mg; 0.01 mg to 525 mg; 0.01 mg to 550 mg; 0.01 mg to 575 mg; 0.01 mg to 600 mg; 0.01 mg to 625 mg; 0.01 mg to 650 mg; 0.01 mg to 675 mg; 0.01 mg to 700 mg; 0.01 mg to 725 mg; 0.01 mg to 750 mg; 0.01 mg to 775 mg; 0.01 mg to 800 mg; 0.01 mg to 825 mg; 0.01 mg to 950 mg; 0.01 mg to 875 mg; 0.01 mg to 900 mg; 0.01 mg to 925 mg; 0.01 mg to 950 mg; 0.01 mg to 975 mg; 0.01 mg to 1000 mg; 0.01 mg to 2000 mg; 0.01 mg to 3000 mg; 0.01 mg to 4000 mg; 01 mg to 5000 mg; 0.01 mg to 0.1 mg/kg; 0.01 mg to 0.5 mg/kg; 01 mg to 1 mg/kg; 0.01 mg to 5 mg/kg; 0.01 mg to 10 mg/kg; 0.01 mg to 25 mg/kg; 0.01 mg to 50 mg/kg; 0.01 mg to 75 mg/kg; and 0.01 mg to 100 mg/kg.

Implementation may generally involve identifying patients suffering from the indicated disorders and administering the compounds of the present invention in an acceptable form by an appropriate route. The exact dosage to be administered may vary depending on the age, gender, weight, and overall health status of the individual patient, as well as the precise etiology of the disease. However, in general, for administration in mammals (e.g., humans), dosages in the range of from about 0.01 to about 300 mg of compound per kg of body weight per 24 hr, and more preferably about 0.01 to about 100 mg of compound per kg of body weight per 24 hr, may be effective. Administration may be oral or parenteral, including intravenously, intramuscularly, subcutaneously, intradermal injection, intraperitoneal injection, etc., or by other routes (e.g., transdermal, sublingual, oral, rectal, and buccal delivery, inhalation of an aerosol, etc.). In a preferred embodiment of the invention, the nanoencapsulated complexes are provided orally or intravenously.

The compounds may be administered in the pure form or in a pharmaceutically acceptable formulation including suitable elixirs, binders, and the like (generally referred to as a “secondary carrier”) or as pharmaceutically acceptable salts (e.g., alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil-based vehicles. Water may be used as the carrier for the preparation of compositions (e.g., injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc.). Solid diluents and excipients include lactose, starch, conventional disintergrating agents, coatings, and the like. Preservatives such as methyl paraben or benzalkium chloride may also be used. Depending on the formulation, it is expected that the active composition will consist of about 1% to about 99% of the composition and the secondary carrier will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, bolus dose, or at a gradual or continuous, constant, or controlled rate to a patient. In addition, the time of day and the number of times per day that the pharmaceutical formulation is administered may vary and are best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

The present inventors sought to determine if the solubility of Cordyceps, Reishi & Lion's Mane extracts would increase with the presence of TPGS in an aqueous solution. For the Cordyceps samples, Ultra Performance Liquid Chromatography (UPLC) analysis was used to quantify Cordycepin, an active component in Cordyceps extract, in the pure aqueous & TPGS solutions. For Reishi & Lion's Mane, UPLC analysis was used to identify the overall content. The solubility of Cordyceps, Reishi & Lion's Mane extracts in water and 20% TPGS solution was analyzed at varying mass ratios of extract to d-α-tocopheryl, the active compound in TPGS mixture. While TPGS did not increase the solubility of Cordycepin in Cordyceps extract, it did increase the overall solubility of Cordyceps, Reishi & Lion's mane extracts. Moreover, while the TPGS did not appear to increase the individual solubility of Psilocybin compound in extract, it did increase the overall solubility of Psilocybin extracts.

### Example 1: Preparation of Cordyceps and Reishi Extracts and TPGS Samples

As shown in Table 1 below, the present inventors evaluated the ability of TPGS to increase the solubility of Cordyceps and Reishi extracts. For the Cordyceps & Reishi test samples, varying mass ratios of extract to active TPGS component were investigated. The designated amount was combined with 50 ml of TPGS stock solution at 50° C. The solution was manually shaken for 15 seconds, vortexed for 15 seconds, sonicated for 60 minutes at 50° C. and allowed to cool at 20° C., protected from light. After 4 hours, the samples were stored at 4° C. This process was repeated twice more. For the control samples, the designated amount of extract was combined with 50 ml pure water, and the below procedure was followed.

TABLE 1
The mass amounts of extracts used for
the Cordyceps & Reishi solutions.

| | Volume | Mass active | |
|---|---|---|---|
| | TPGS 20% | TPGS | Mass |
| | solution | component | extract |
| Ratio | [ml] | [mg] | [mg] |
| 30:1 | 50 | 2690 | 90 |
| 25:1 | 50 | 2690 | 108 |
| 20:1 | 50 | 2690 | 135 |
| 15:1 | 50 | 2690 | 179 |

The preparation of a TPGS stock solution included: TPGS (20 g) mixed with LCMS grade water (800 ml) in a 1 L media bottle. The mixture was stirred gently on a magnetic stir-plate at ° C. for 4 hours. This method was used to produce 2 L of stock solution. The stock solutions was stored at 4° C.

### Example 2: Preparation of Lion's Mane Extract and TPGS Samples

As shown in Table 2 below, the present inventors evaluated the ability of TPGS to increase the solubility of Lion's Mane extract (also referred to as Hericium erinaceum extract or an extract of Hericium erinaceum). Designated amounts of Lion's Mane extract aqueous suspension were combined with pure water to a volume of 1 ml and added to 1 ml saturated TPGS solution. After, the same testing and control procedures were followed as described in Example 1 above.

TABLE 2
The mass amounts of extracts used for
the Cordyceps & Reishi solutions.

| Lion's | Volume | Volume |
|---|---|---|
| Mane extract | pure water | TPGS 20% |
| suspension [ml] | [ml] | solution [ml] |
| 0.25 | 0.75 | 1 |
| 0.5 | 0.5 | 1 |
| 0.75 | 0.25 | 1 |
| 1.00 | 0 | 1 |

### Example 3: UPLC Analysis of Extract and TPGS Samples

For UPLC analysis, 1 ml of each solution was removed from the bulk, placed in a 1 ml microcentrifuge tube and centrifuged for 5 minutes at 6000 rpms. The supernatant was removed, filtered through a 0.22-micron filter and placed in an amber UPLC vial. This method was applied to the Cordyceps, Reishi & Lion's Mane Samples. The TPGS samples were sonicated for 5 minutes in a 50° C. bath and promptly placed in the UPLC tray just before the sample was drawn. The purpose of this was to disrupt the micelles for an accurate analysis. The control samples were simply placed in the UPLC.

### Example 4: Preparation of Psilocybin and TPGS Samples

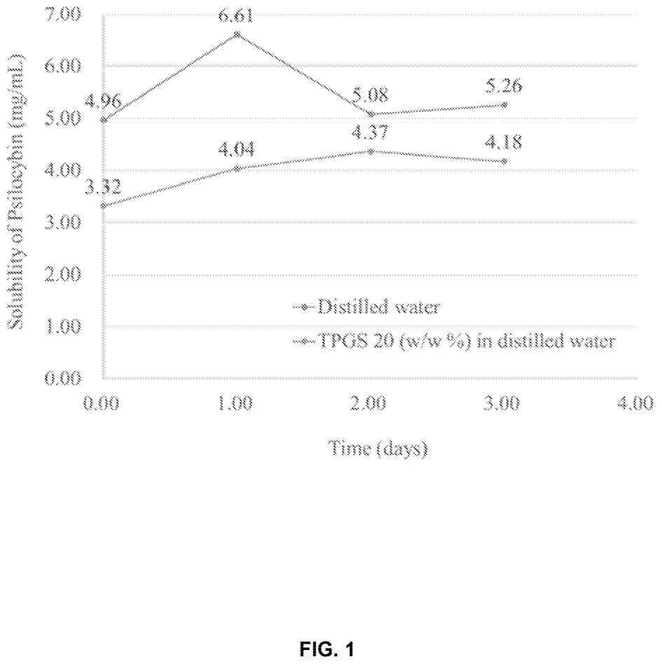

As shown in FIG. 1, the present inventors evaluated the ability of TPGS to increase the solubility of psilocybin extract. A psilocybin/TPGS sample was generated by the present inventors by the following process: Psilocybin (40 mg) was dissolved in 4 ml distilled water in a 15 ml centrifuge tube. Psilocybin (40 mg) was dissolved in 4 ml TPGS stock solution in another 15 ml centrifuge tube. Both samples were sonicated for 20 min and were incubated at 25° C. on a shaker in the dark (shaking was set at low speed). A small quantity (0.5 ml) from both the samples was transferred to a small Eppendorf tube and centrifuged for 10 minutes at 13,000 rpms. The samples were analyzed by the validated UPLC method at 0 h (initial), 24 h, 48 h and 72 h and psilocybin content were calculated (mg/ml). Solubility of Psilocybin was increased through the addition of TPGS as shown in FIG. 1.

A TPGS stock solution was generated by the present inventors by the following process: TPGS (20 g) was mixed with LCMS grade water (80 g) in a 250 ml media bottle. The mixture was stirred gently on a magnetic stir-plate at 50° C. for 4 hours. The stock solutions was stored at 4° C.

### Example 5. Solubility Analysis of Extract and TPGS Samples

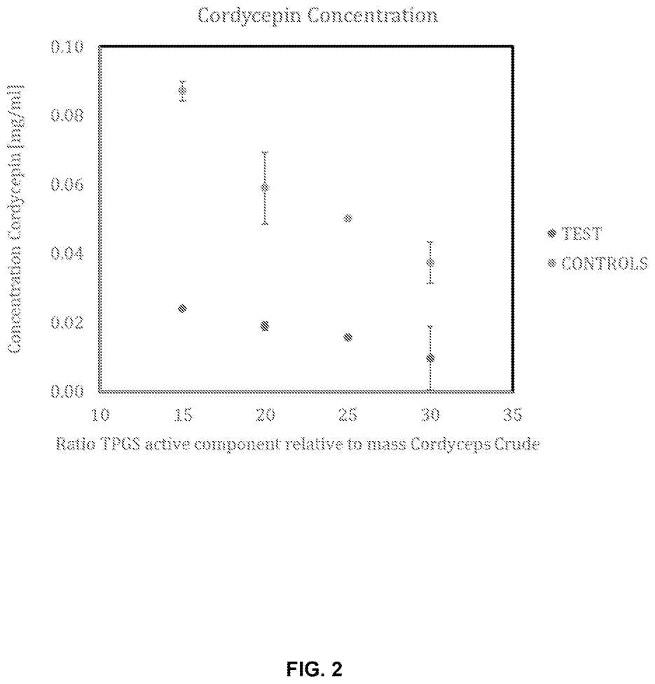

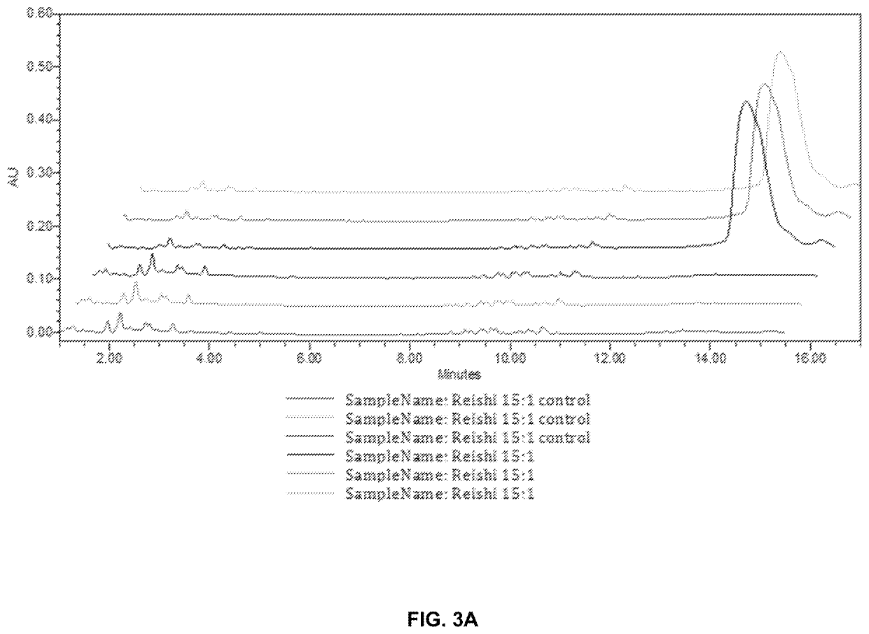

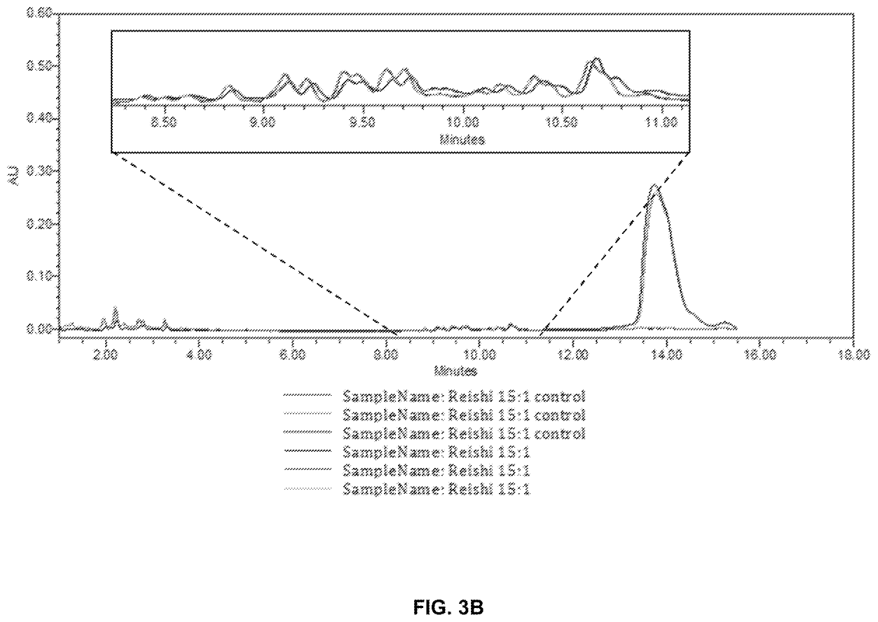

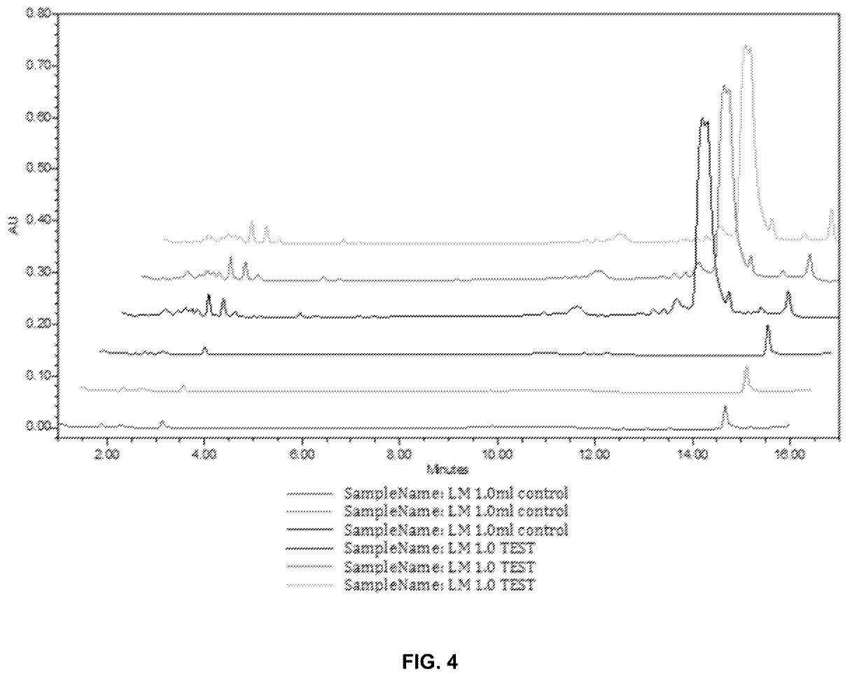

The present inventors evaluated the solubility of the exemplary extracts and TPGS samples. As shown in FIG. 2, cordycepin extracts appeared to be more soluble in pure water than the saturated TPGS solution. As further shown in FIGS. 3A and 3B, TPGS increased the solubility of the Reishi extract in one embodiment. As further shown in FIGS. 4A and 4B, TPGS increased the solubility of the Lion's Mane extract in one embodiment. Notably, no single component was identified & quantified with respect to the Reishi & Lion's Mane extract samples. Instead, the overall absorbance was compared via chromatograms.

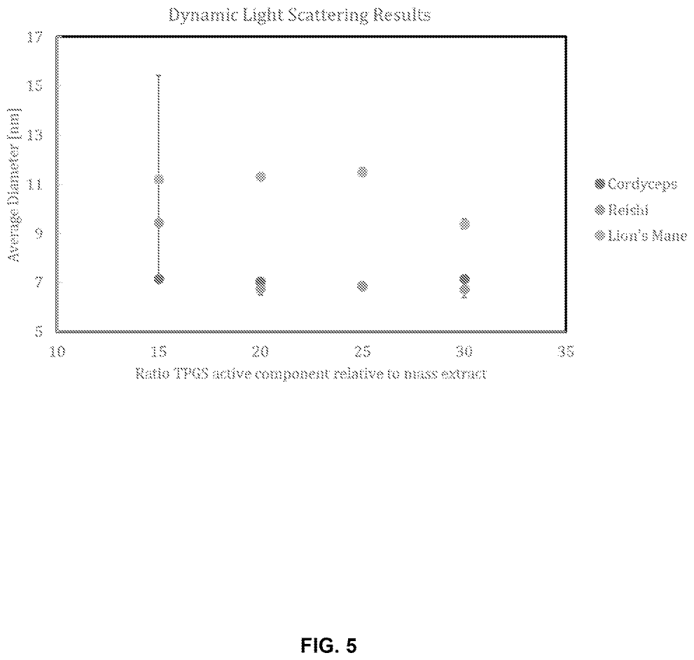

The present inventors took diameter measurement via Dynamic Light Scattering (DLS) for the Cordyceps, Reishi & Lion's mane extracts samples. As shown in FIG. 5, the diameter of micelles appears to be independent of mass ratio. Notably, the diameter of all samples is homogenous and below the 20 nm threshold suggesting an increase in bioavailability and mucosal membrane permeability.

As shown in the data presented herein, it was observed that the existence of TPGS in aqueous solution appears to increase loading for Lion's Mane & Reishi samples. Concerning Psilocybin and Cordycepin extracts, TPGS may not increase loading of the specific compounds, but may increase the loading of other compounds within the Cordyceps and Psilocybin extracts increasing solubility and bioavailability of the extract components.

### Definitions

The term “fungal culture” as used herein means a culture representing one or more fungal species, which may be produced by any suitable method including, but not limiting to, sequential translocation of the target fungal mycelium on growth medium to isolate a single fungal strain from a surface sterilized root material of woody plants as a pure culture.

The term “extract” as used herein means a substance obtained by any extraction method, whether or not the crude extract has been fractioned or purified by usual methods such as chromatography or filtration. Extraction methods include, but are not limited to pressing and solvent extraction, and any suitable solvent may be used. The solvent may be an organic or inorganic solvent, or a mixture of suitable solvents. Typically, deionized water or a phosphate balanced buffer (PBS), pH 7.4 is used as an inorganic solvent, and an aqueous ethanol mix is used as the organic solvent. However, also other inorganic and organic solvents may be used. The extraction solvent may also be a supercritical fluid such as supercritical water or CO₂. The term fungal extract includes those obtained from fungal cultures of single or multiple fungal species or strains, as well as mixtures of said extracts.

As used herein, a “compound” derived from a fungus or “fungal extract”, and any equivalents thereof, refers to one or more isolated and/or purified compounds derived from said extract, and/or compounds present in a purified or crude extract. The term also refers to synthetic counterparts of the compounds found in said extracts as well as to said compounds produced in genetically modified organisms other than the original fungi. All compounds of interest referred to herein explicitly encompass all forms of such compounds, such as and salts, hydrates, and solvates thereof, as well as all zwitterionic, tautomeric, stereoisomeric, and enantiomeric forms.

As used herein, a compound, or compounds of the invention are referred to as “isolated” or “purified” when it has been separated from at least one component with which it is naturally associated. For example, a compound, such as Psilocybin or psilocin can be considered isolated if it is separated from contaminants of the compounds or cellular or artificial components. Isolated or purified compounds can be either prepared synthetically or purified from their natural environment. Standard quantification methodologies known in the art can be employed to obtain and isolate the molecules of the invention. Within the context of this disclosure, purified compounds may be purposely formulated with other compounds at various levels of purity. For example, depending on the desired outcome, a particular compound derived from fungal extract may be formulated with other molecules when it is 60-65% pure, 65-70% pure, 70-75% pure, 75-80% pure, 80-85% pure, 85-90% pure, 90-95% pure, 95-99% pure, 99-99.9% pure, 99.9+%, or greater than 99% pure. Provided that the ingredients used for purposeful formulation are purified prior to the said purposeful formulation, the act of subsequently formulating them does render them not “purified” within the context of an ingredient list.

As used herein, a compound, or compounds or extract, or extracts of the invention are referred to as “derived from” a source when they are isolated or separated from at least one component with which it is naturally associated. For example, a compound, such as Psilocybin or psilocin can be considered derived if it is separated from contaminants of the compounds or cellular or artificial components. Derived extracts can include whole or partial extracts isolated from a fungal feedstock. In some embodiment, an extract may be derived from a portion of a fungal feedstock, such as a soluble or insoluble fraction.

As used herein, the terms “solubilizing,” “solubilize,” a compound or having a “solubilizing effect” on such compound of the invention shall mean having the effect of increasing the solubility in water of the compound. In a preferred embodiment, the effect of increasing the solubility in water of the compound comprises an increase of at least about two-fold (i.e., reducing by at least about half the amount of water required to dissolve one gram of the compound). As used herein, the term “soluble” or “water soluble” refers to a compound or compounds dissolvable in water or liquid. In one embodiment, water soluble comprises dissolving a compound in water. In one embodiment, dissolving comprises heating. In one embodiment, dissolving comprises stirring. In one embodiment, dissolving comprises shaking. In one embodiment, dissolving comprises mixing. In one embodiment, a powder is water soluble. In one embodiment, a composition is water soluble, and preferably a compound contained within a nanoencapsulated complex.

As used herein, the terms “increased bioavailability,” “increasing bioavailability,” or “enhanced bioavailability” of one or more compound(s) of the invention administered to a subject shall mean, in reference to the effect of administering one or more compounds of the invention in a solubilized nanoemulsion complex that results in an increase in the portion of the dose of the compound(s) administered that reaches one or more targeted systemic fluids, organs, tissues, or cells as compared to administration without the solubilized nanoemulsion complex, and preferably a TPGS solubilized nanoemulsion complex. Increased bioavailability can include any mechanism that that has a desired effect on cellular efflux, cellular influx, or clearance. “Clearance” includes any type of elimination of one or more compounds from cells, blood, plasma, tissues, or organs (e.g., intestinal clearance, hepatic clearance, renal clearance, and pulmonary clearance each describe elimination of compounds from the blood). Clearance may be described via the observed differences of renal excretion and elimination by all other processes including influx and efflux mechanisms (e.g., gastrointestinal clearance, excretory clearance, biliary clearance and enterohepatic cycling, metabolic clearance). Examples of systemic fluids include, but are not limited to: blood; cerebrospinal fluid; lymph; and any other tissue fluids (including increased amounts in tissues that are bathed by such fluids, such as the brain, tissue of one or more visceral organs, connective tissue, muscle, fat, or one or more tissues in the skin). In some embodiments, the increase is systemic, as in the case of an increase measurable anywhere in the blood. In some embodiments, the increase is more localized, as is the case with some embodiments involving topical administration in which the increase is measured only in areas near the administration. An increase in portion of the dosage that reaches a fluid or tissue measurable by any reliable means is within this definition, including but not limited to increases identified by measuring the total systemic compound concentration over time after administration. In some embodiments, concentrations are determined by measuring the tissue or fluids themselves, or by measuring fractions thereof (for example, without limitation, serum, or plasma in the case of blood). In some embodiments, increases for compounds that are excreted metabolized and/or un-metabolized in urine are determined by measuring levels of compounds or metabolites of the compounds in urine and will reflect an increase in systemic concentrations. In some embodiments an increase in compound bioavailability is defined as an increase in the Area Under the Curve (AUC). AUC is an integrated measure of systemic compound concentrations over time in units of mass-time/volume and is measured from the time compound is administered (time zero) to infinity (when no compound(s) remaining in the body can be measured). Information regarding monitoring substances are known to persons of ordinary skill in the art.

In one embodiment, “increased bioavailability,” “increasing bioavailability,” or “enhanced bioavailability” of one or more compound(s) of the invention may include a compound or extract component having a quantity of TPGS having a diameter, and in particular a micelle of 20 nm or less indicating and increase in bioavailability and mucosal membrane permeability.

As used herein, the term “contained within,” “containing,” or “having” refers to molecules, e.g., one or more compounds of the invention, that are sequestered inside a spherical shape formed by micelles and reverse micelles. In one embodiment, a compounds of interest contained within a micelle allows said compound to disperse or dissolve within an aqueous formulation. As used herein, the term “micelle” refers to a collection of molecules arranged alongside one another in a spherical form often having a pocket inside. In one embodiment, the micelle comprises a lipid molecule. In one embodiment, the lipid molecule comprises both a hydrophobic and hydrophilic region. In one embodiment, the micelle is in a solvent. In one embodiment, the hydrophilic region is in contact with surrounding solvent, sequestering the hydrophobic region in the micelle center. In one embodiment, the micelle is in water and the polar group is on the outside and a hydrophobic end sequesters inside the spherical shape. In one embodiment, the micelle is a reverse micelle, i.e., the hydrophilic region of a molecule is surrounded by a nonpolar solvent resulting in a water in oil system. In one embodiment, the reverse micelle comprises hydrophobic groups extended away from the center while hydrophilic groups are sequestered inside the spherical shape. In a preferred embodiment, a “micelle” is formed by Vitamin E bioavailability and mucosal membrane permeability. In a preferred embodiment, a micelle may be at or below 20 nm in diameter.

As described herein, in one or more embodiment one or more compounds of the invention may be solubilized in a nanoemulsion complex and administered to a human or animal under conditions effective to cause absorption to the bloodstream, or into target cells, tissues, or organs, causes a therapeutic or prophylactic effect. In a preferred embodiment, the amount of solubilized in a nanoemulsion complex containing one or more active compounds of interest may be a therapeutically effective amount.

As used herein, the term “percent mass” refers to the amount of matter of a compound expressed as a fraction of 100. In one embodiment, the percent mass is expressed in grams. In one embodiment, the percent mass is expressed in ounces. In one embodiment, the percent mass is expressed in moles. In one embodiment, the percent mass is the amount of a first purified compound or extract in a composition. In one embodiment, the percent is the amount of solubilizing excipient, such as Vitamin E TPGS in a composition. In one embodiment, the percent mass is calculated with the following formula: mass of compound+total mass of sampler 100.

The terms “approximately” and “about” refer to a quantity, level, value, or amount that varies by as much as 30%, or in another embodiment by as much as 20%, and in a third embodiment by as much as 10% to a reference quantity, level, value, or amount. As used herein, the singular form “a,” “an,” and “the” include plural references unless the context clearly dictates otherwise.

As used herein, the term “ratio” refers to the relative amount of one or more compounds in relation to another compound or compounds. In one embodiment, the ratio is in reference to the mass of one compound to another. In one embodiment, the ratio is in reference to the mass percent of one compound to another. In one embodiment, the ratio is in reference to the dry weight of one compound to another. In one embodiment, the ratio is in reference to the volume of one compound to another. In one embodiment, the ratio is in reference to the molar mass of one compound to another.

1. A pharmaceutical composition comprising:
a quantity of psilocybin or psilocin;
a quantity of d-a-tocopheryl polyethylene glycol succinate (TPGS), wherein said psilocybin or psilocin and said TPGS form a nanoencapsulated complex; and
a pharmaceutically acceptable carrier.

2. The composition of claim 1, wherein said quantity of psilocybin or psilocin comprises an fungal extract of interest from a fungal feedstock containing psilocybin or psilocin.

3. The composition of claim 2, wherein said fungal feedstock comprises a psilocybin mushroom fungal feedstock.

4. (canceled)

5. The composition of claim 1, wherein said quantity of psilocybin or psilocin comprises an isolated quantity of psilocybin or psilocin.

6. The composition of claim 1, wherein said quantity of psilocybin or psilocin comprises an isolated quantity of chemically synthesized psilocybin or psilocin.

7. The composition of claim 1, wherein said nanoencapsulated complex is at least or less than 20 nm in diameter.

8. The composition of claim 1, wherein said pharmaceutically acceptable carrier comprises an aqueous solution.

9. The composition of claim 8, wherein said aqueous solution comprises a sonicated aqueous solution.

10. The composition of claim 1, wherein said nanoencapsulated complex is isolated from said aqueous solution.

11. The composition of claim 1, wherein said pharmaceutically acceptable carrier comprises a solubilizable dry powder.

12-13. (canceled)

14. A pharmaceutical composition comprising:
a compound or fungal extract of interest derived from a fungal feedstock;
a quantity of TPGS, wherein said compound or fungal extract of interest and said TPGS form a nanoencapsulated complex; and
a pharmaceutically acceptable carrier.

15. The composition of claim 14, wherein said compound of interest derived from a fungal feedstock comprises psilocybin or psilocin, or a combination of the same.

16-17. (canceled)

18. The composition of claim 14, wherein said fungal extract of interest derived from a fungal feedstock comprises one or more fungal extracts selected from the group consisting of: a Ganoderma sp. extract, a Cordyceps sp. extract, a Psilocybe sp. extract, a Hericium sp. extract, a Chaga sp. extract, or a combination of the same.

19. The composition of claim 14, wherein said fungal extract of interest derived from a fungal feedstock comprises one or more fungal extracts selected from the group consisting of: a Ganoderma lucidum extract, a Cordyceps militaris extract, a Psilocybe sp. extract, a Hericium erinaceum extract, and a Chaga sp. extract, or a combination of the same.

20. The composition of claim 14, wherein said fungal extract of interest derived from a fungal feedstock comprises one or more fungal extracts selected from the group consisting of: a cordyceps extract, a reishi extract, a lion's mane extract, and a psilocybin mushroom extract.

21. The composition of claim 14, wherein said nanoencapsulated complex is at least or less than 20 nm in diameter.

22. The composition of claim 14, wherein said pharmaceutically acceptable carrier comprises an aqueous solution.

23. The composition of claim 22, wherein said aqueous solution comprises a sonicated aqueous solution.

24. The composition of claim 14, wherein said nanoencapsulated complex is isolated from said aqueous solution.

25. The composition of claim 14, wherein said pharmaceutically acceptable carrier comprises a solubilizable dry powder.

26-113. (canceled)
